(19) United States
(12) Patent Application Publication
(10) Pub. No.: US 2024/0382753 A1
(43) Pub. Date: Nov. 21, 2024

(54) METHODS FOR VECTOR-BASED TARGETING OF THE HUMAN CENTRAL THALAMUS TO GUIDE DEEP BRAIN STIMULATION AND DEVICES THEREFOR

(71) Applicants: CORNELL UNIVERSITY, Ithaca, NY (US); THE UNIVERSITY OF UTAH, Salt Lake City, UT (US); THE BOARD OF TRUSTEES OF THE LELAND STANFORD JUNIOR UNIVERSITY, Stanford, CA (US)

(72) Inventors: Nicholas SCHIFF, New York, NY (US); Jonathan BAKER, Scarsdale, NY (US); Christopher BUTSON, Salt Lake City, UT (US); Andrew JANSON, Salt Lake City, UT (US); Kyle O'SULLIVAN, Stanford, CA (US); Jaimie HENDERSON, Stanford, CA (US); Eun Young CHOI, Stanford, CA (US); Brian RUTT, Stanford, CA (US); Matthew RADOVAN, Stanford, CA (US); Jason SU, Stanford, CA (US)

(21) Appl. No.: 18/692,517

(22) PCT Filed: Sep. 14, 2022
(86) PCT No.: PCT/US2022/043451

§ 371 Date: Mar. 15, 2024

Related U.S. Application Data
(60) Provisional application No. 63/244,589, filed on Sep. 15, 2021.

(51) Int. Cl. A61N 1/36; A61N 1/05

(52) U.S. Cl. CPC A61N 1/36082; A61N 1/0534; A61N 1/36171

(57) ABSTRACT

Methods and devices for vector-based targeting of the human central thalamus (CT) to guide deep brain stimulation (DBS) are disclosed. In some examples, electrode(s) each with a plurality contacts are provided. A three-dimensional orientation of a dominant axis of a central lateral nucleus dorsal tegmental tract medial component (CL/DTTm) fiber bundle of a human subject is determined. The contacts of the electrode(s) are positioned in the subject's CT fibers in substantial alignment with the three-dimensional orientation. An electrical stimulus is applied to the contacts to selectively activate the CT fibers. The positioning and the applying are carried out to maximize activation of a central lateral nucleus and medial dorsal tegmental tract fiber pathway in the subject and to minimize activation of a centromedian-parafascicularis fiber pathway in the subject. Methods and devices for surgical planning involving for vector-based targeting of the human CT to guide DBS are also disclosed.

This application claims benefit of U.S. Provisional Patent Application No. 63/244,589, filed Sep. 15, 2021, the entirety of which is incorporated herein by reference.

This invention was made with government support under grant number UH3 NS095554 awarded by National Institute of Health-National Institute of Neurological Disorders and Stroke. The government has certain rights in this invention.

### FIELD

The present technology relates to methods and devices, including systems and non-transitory computer readable media, for vector-based targeting of the human central thalamus to guide deep brain stimulation.

### BACKGROUND

The central thalamus (CT) is a key node in the arousal regulation network of the mammalian brain hypothesized to modulate large-scale activity patterns across the anterior forebrain in response to internal and external demands during wakefulness. Damage of the CT in humans, due to traumatic brain injury (TBI) or stroke, for example, results in enduring cognitive deficits in the allocation of attention, maintenance of concentration and focus, working memory, impulse control, processing speed, and motivation. Moreover, due to the geometrical properties of neurons within the CT, which demonstrate wide point-to-point connections across the cortico-thalamic system and to the striatum, impaired cognition (typically in the form of dysexecutive function) arousal regulation, due to loss of neurons within the CT, is a common consequence of multi-focal brain injuries typical of traumatic brain injuries, anoxia, hypoxic-ischemic encephalopathy, multi-focal ischemic injuries as sustained due to vasospasm (from e.g. aneurysmal hemorrhage, vasculitis, other causes), or a wide range of toxic-metabolic, post-infectious, auto-immune, or other causes.

As current therapeutics are not effective at treating these cognitive deficits, deep brain stimulation (DBS) within the central thalamus (CT-DBS) has been proposed as a therapeutic option to artificially restore arousal regulation in order to reestablish and/or broadly support cognitive function in TBI subjects. By targeting the ‘wing’ of the central lateral (CL) nucleus, and its projecting fiber bundle of axons, CT-DBS can result in a significant and cumulative improvement in a subject's responsiveness, communication, and motor function following a very severe TBI. However, the mechanisms that produce this outcome, which are dependent on DBS lead (e.g., electrodes and/or associated contacts) location and methods of neural activation, remain unknown.

The use of DBS to treat very severe TBI subjects has a long history of failure, primarily due to poor subject selection and hypothesis-free DBS targeting. The predominant target for DBS in these subjects has been the centromedian-parafascicularis complex (Cm-Pf) of the thalamus, a relatively large and prominent nucleus adjacent to the CL nucleus. Yet to date, clinical outcomes in this subject population have been highly variable due to several factors such as the etiology of subjects investigated, the ability to successfully target and acquire CM-Pf during lead implantation, the background spontaneous recovery rate from TBIs within the first year following an injury.

Despite the variability in clinical results in very severe brain injuries, the preclinical evidence for enhancing arousal and behavioral performance in intact animals during electrical stimulation of CL is more extensive. Recent studies confirm that electrical stimulation of CL can effectively enhance arousal and performance in healthy rodents and in two rodent models of pathology, epilepsy and TBI. In anesthetized animals, optogenetic stimulation of CL in mice and electrical stimulation of CL in rodents and non-human primates (NHP) demonstrate broad cortical and subcortical activations.

A recent study in healthy behaving non-human primates (NHPs) expanded on these results, examining the effects of various methods of CT-DBS on behavior and physiology while the animals performed more complex visuomotor tasks. A unique aspect of this study was the use of two closely spaced DBS leads placed within the CT and the discovery that both the precise location of the leads in CT and the orientation of the electric field established between the two leads were critical parameters for improving performance and enhancing frontostriatal activity patterns.

More recent work has determined that locating DBS electrodes so as to maximize central lateral nucleus and medial dorsal tegmental tract (CL/DTTm) fiber pathway activation in a subject, and to minimize centromedian-parafascicularis fiber pathway activation in the subject, resulted in advantageous outcomes, as explained in U.S. Pat. No. 9,592,383 and PCT application Serial No. PCT/US2021/023648, each of which is hereby incorporated by reference in its entirety. In this work, field-shaping within the central thalamus (fsCT-DBS) utilizing at least two stimulators to control a thalamic fiber was used to selectively target activation and target avoidance in the mammalian thalamus, which was reduced to practice in direct measurements from experiments carried out in non-human primates. Thus, by applying CT-DBS to subjects with moderate-to-severe traumatic brain injuries, improved arousal regulation has been shown to correlate with activation of CL/DTTm.

The present application is directed to further enhancing deep brain stimulation techniques.

### SUMMARY

In some aspects, the disclosed technology relates to human central thalamic targeting to achieve target activation and successful target avoidance of regions of human intra-thalamic pathways within the central thalamus to achieve vector-based placement of deep brain stimulation electrodes. In some examples, this technology facilitates target acquisition and avoidance of human intra-central thalamic pathways in human subjects based on imaging, thalamic segmentation protocols, and predictive biophysical models that estimate activation of projection fibers to accurately determine a vector corresponding to a dominant axis of a central lateral nucleus dorsal tegmental tract medial component (CL/DTTm) fiber bundle and locate deep brain stimulation (DBS) electrode contacts in substantial alignment with the determined vector and/or the dominant axis.

One aspect of the present technology relates to a method for vector-based targeting of a human central thalamus to guide deep brain stimulation (DBS). The method involves providing one or more electrodes each with a plurality of contacts. A three-dimensional orientation of a dominant axis of a CL/DTTm fiber bundle of the human subject is determined. The plurality of contacts of the one or more electrodes are then positioned in the human subject's central thalamus fibers in substantial alignment with the determined three-dimensional orientation of the dominant axis of the CL/DTTm fiber bundle. An electrical stimulus is then applied to the positioned plurality of contacts of the one or more electrodes to treat the human subject for impaired arousal regulation. The positioning and the applying are carried out to maximize activation of a central lateral nucleus and medial dorsal tegmental tract fiber pathway in the human subject and to minimize activation of a centromedian-parafascicularis fiber pathway in the human subject.

Another aspect of the present technology relates to a method of treating a condition characterized by impaired arousal regulation in a human subject. The method involves selecting a human subject with impaired arousal regulation. One or more electrodes are provided each with a plurality of contacts. A three-dimensional orientation of a dominant axis of a CL/DTTm fiber bundle of the human subject is determined. The plurality of contacts of the one or more electrodes are then positioned in the human subject's central thalamus fibers in substantial alignment with the determined three-dimensional orientation of the dominant axis of the CL/DTTm fiber bundle. An electrical stimulus is then applied to the positioned plurality of contacts of the one or more electrodes to selectively activate the central thalamus fibers of the human subject. The positioning and the applying are carried out to maximize activation of a central lateral nucleus and medial dorsal tegmental tract fiber pathway in the human subject and to minimize activation of a centromedian-parafascicularis fiber pathway in the human subject.

A further aspect of the present technology relates to a method for surgical planning involving vector-based targeting of a human central thalamus to guide DBS implemented by one or more surgical computing devices. The method involves segmenting the central thalamus in an image of a bran of the human subject to produce a segmented brain model. One or more fiber pathways in the segmented brain model are modeled. A three-dimensional orientation of a dominant axis of a CL/DTTm fiber bundle of the human subject is determined based on the modelling. Initial model positions and orientations in the segmented brain model are generated for one or more electrodes based at least in part on the determined three-dimensional orientation of the dominant axis of the CL/DTTm fiber bundle of the human subject. A stimulation map is produced based on the modelling and the generating. A position and orientation for a plurality of contacts of the one or more electrodes in the human subject's central thalamus fibers and electrical stimulus conditions for the positioned and oriented plurality of contacts of the one or more electrodes are identified to selectively activate the central thalamus fibers of the human subject. This permits activation of a central lateral nucleus and medial dorsal tegmental tract fiber pathway in the human subject is maximized and activation of a centromedian-parafascicularis fiber pathway in the human subject is minimized based on the produced simulation map.

Yet another aspect of the present technology relates to a non-transitory computer readable medium having stored thereon instructions for surgical planning involving vector-based targeting of a human central thalamus to guide DBS. The non-transitory computer readable medium includes executable code that, when executed by one or more processors, causes the one or more processors to segment the central thalamus in an image of the human subject's brain to produce a segmented brain model. One or more fiber pathways in the segmented brain model are modeled. A three-dimensional orientation of a dominant axis of a CL/DTTm fiber bundle of the human subject is determined based on the modelling. Initial model positions and orientations in the segmented brain model are generated for one or more electrodes based at least in part on the determined three-dimensional orientation of the dominant axis of the CL/DTTm fiber bundle of the human subject. A stimulation map is produced based on the modelling and the generating. A position and orientation for a plurality of contacts of the one or more electrodes in the human subject's central thalamus fibers and electrical stimulus conditions for the positioned and oriented plurality of contacts of the one or more electrodes are identified to selectively activate the central thalamus fibers of the human subject so that activation of a central lateral nucleus and medial dorsal tegmental tract fiber pathway in the human subject is maximized and activation of a centromedian-parafascicularis fiber pathway in the human subject is minimized based on the produced simulation map.

Another aspect of the present technology relates to a surgical computing device. The surgical computing device includes comprising memory comprising programmed instructions stored thereon and one or more processors coupled to the memory and configured to execute the stored programmed instructions. The stored programmed instructions include segmenting the central thalamus in an image of a bran of the human subject to produce a segmented brain model. One or more fiber pathways in the segmented brain model are modeled. A three-dimensional orientation of a dominant axis of a CL/DTTm fiber bundle of the human subject is determined based on the modelling. Initial model positions and orientations in the segmented brain model are generated for one or more electrodes based at least in part on the determined three-dimensional orientation of the dominant axis of the CL/DTTm fiber bundle of the human subject. A stimulation map is produced based on the modelling and the generating. A position and orientation for a plurality of contacts of the one or more electrodes in the human subject's central thalamus fibers and electrical stimulus conditions for the positioned and oriented plurality of contacts of the one or more electrodes are identified to selectively activate the central thalamus fibers of the human subject so that activation of a central lateral nucleus and medial dorsal tegmental tract fiber pathway in the human subject is maximized and activation of a centromedian-parafascicularis fiber pathway in the human subject is minimized based on the produced simulation map.

A further aspect of the present technology relates to a system for vector-based targeting of a human central thalamus to guide DBS. The system includes the surgical computing device of the present technology. The system also includes an imaging device operationally coupled to the surgical planning system and one or more electrodes. An electrical stimulator is coupled to the surgical computing device and the one or more electrodes to permit electrical activation of the electrodes based on instructions from the surgical computing device.

The present technology advantageously provides methods of treatment and systems that enable treatment via vector-based targeting of a human central thalamus to guide DBS to support forebrain arousal regulation via the activation of fibers emanating from the central lateral nucleus of the central thalamus (CL) and the surrounding dorsal tegmental track medial (DTTm). The CL/DTTm target can be activated optimally by shaping the applied electrical field by utilizing one or more leads, or stimulators, with many electrode contacts place in substantial alignment with an orientation of a dominant axis of the CL/DTTm fiber bundle determined from fiber pathway modeling.

Key targets for stimulation are the local fiber tracts that traverse the CT, such as the medial dorsal tegmental tract (DTTm), a component of the ascending reticular activating system that passes through CL and into the thalamic reticular nucleus (TRN) that in turn projects broadly to the cortex and striatum. The DTTm also carries glutamatergic efferents from the CL nucleus to the TRN, cortex, and striatum. A precise therapeutic DBS target may be difficult to determine for many TBI subjects given the presence of a wide range of structural injuries in this population including substantial deformation and atrophy of the thalamic nuclei. However, subjects with higher levels of consciousness and less structural injury of their thalamus, frontal lobe, and striatum are expected to be ideal candidates for DBS therapy as they often suffer from enduring cognitive dysfunction. In such persons, however, improved targeting and activation of the arousal related pathways that minimizes OFF-target side effects, are critical to developing this potential therapy, as recently demonstrated. The DTTm fiber pathway is an optimal DBS target to facilitate performance in healthy NHPs, which directly informs ongoing and future clinical studies using DBS to treat the enduring fatigue and cognitive dysfunction experienced by the majority TBI subjects. The technology described and illustrated herein improves the activation of target regions of the CL/DTTm fiber bundle based on substantially aligning an orientation of contacts of inserted electrodes with a dominant axis of the CL/DTTm fiber bundle.

Central thalamic deep brain stimulation (CT-DBS) is an investigational therapy to treat enduring cognitive dysfunction in humans following traumatic brain injury (TBI). However, the mechanisms of CT-DBS that could promote restoration of cognitive functions are unknown and the heterogeneous etiology and recovery profiles of TBI subjects will likely result in variable outcomes and will be difficult to interpret. Modes of CT-DBS activation of the central thalamus (CT) in healthy non-human primates (NHP) were modeled and experimentally validated as the NHPs performed various visuomotor tasks. Selective activation of a specific fiber pathway, the DTTm and limited activation of the adjacent centromedian-parafascicularis (Cm-Pf) pathway, results in robust behavioral facilitation. The modeling of CT-DBS within these two adjacent thalamic pathways is concordant with the behavioral effects observed across animals. The empirical validation of the biophysical modeling approach in intact behaving NHPs directly informs ongoing and future clinical investigations using conventional and novel modes of CT-DBS in TBI subjects to effectively treat the enduring cognitive dysfunction experienced by the vast majority of these people, for whom no therapy currently exists.

Both CL and Cm-Pf have been reported to be associated with some improved arousal and facilitation of behavior, although the quality of localization in human clinical studies varies making direct comparisons indeterminate. The selective effect of CL/DTTm fibers demonstrated here is consistent with these projections providing a broad excitatory input across frontal cortical and striatal regions. That limited co-activation of the Cm-Pf→TRN fibers limited facilitation, and equal co-activation of these fibers had a suppressive effect, implicates a key role for known anatomical and physiological distinctions between CL neurons and those within the parafascularis (Pf) and centromedian (Cm) nuclei.

Studies of both cortical and striatal activation demonstrate a foundation for the selective behavioral effects associated with CL/DTTm activation. CL/DTTm achieves a very broad activation across frontal cortical (Baker, et al., “Robust Modulation of Arousal Regulation, Performance and Frontostriatal Activity Through Central Thalamic Deep Brain Stimulation in Healthy Non-Human Primates.” J. Neurophysiol. 116:2383-2404 (2016), the disclosure of which is incorporated by reference herein in its entirety) and striatal regions (Liu, et al., “Frequency-Selective Control of Cortical and Subcortical Networks by Central Thalamus. Elife. 4, 1-27 (2015), the disclosure of which is incorporated herein by reference in its entirety) whereas the local microcircuit effects of CL/DTTm and Cm-Pf stimulation within the striatum are distinct. Medium spiny neurons (MSN), the principal output neurons of the striatum, are activated by either CL or Pf afferents but it has been shown that CL afferents are more effective in driving MSN action potentials. Pf afferents, on the other hand, act via NMDA receptors and generate long-term depression through mechanisms of synaptic plasticity (Ellender, et al., “Heterogeneous Properties of Central Lateral and Parafascicular Thalamic Synapses in the Striatum.” J. Physiol. 591, 257-72 (2013), the disclosure of which is incorporated by reference herein in its entirety). These physiological distinctions likely contribute to the reduction of behavioral facilitation that is produced when CL/DTTm and Cm-Pf→TRN fibers are co-activated.

Increased feedback inhibition from the TRN on CL, due to the addition of Cm-Pf→TRN activation, may also contribute to the drop in CL's excitatory effects of frontal lobe function when both pathways are stimulated. Within the neocortex the broad innervation of supergranular and infragranular layers by CL afferents is associated with supralinear summation of effects across cortical columns (Llinás, et al., “Temporal Binding Via Cortical Coincidence Detection of Specific and Nonspecific Thalamocortical Inputs: A Voltage-Dependent Dye-Imaging Study in Mouse Brain Slices.” Proc. Natl. Acad. Sci. U.S. 816 A. 99, 449-454 (2002), the disclosure of which is incorporated herein by reference in its entirety). It is likely the encroachment of activation on Cm-Pf that reduces this selective activation through both local synaptic effects within the striatum (where Cm and Pf innervations are patchy as disclosed in Smith, et al., “The Thalamostriatal Systems: Anatomical and Functional Organization in Normal and Parkinsonian States.” Brain Res. Bull. 78, 60-68 (2009) and Ellender, et al., “Heterogeneous Properties of Central Lateral and Parafascicular Thalamic Synapses in the Striatum.” J. Physiol. 591, 257-72 (2013), the disclosures of which are incorporated by reference herein in their entirety) and powerful inhibition of cell bodies within parts of CL (and paralaminar thalamic regions (Jones, The Thalamus Springer US, Boston, MA, ed. 2nd, (2007) and Münkle, et al., “The Distribution of Calbindin, Calretinin and Parvalbumin Immunoreactivity in the Human Thalamus.” J. Chem. Neuroanat. 19, 155-173 (2000), the disclosure of which are incorporated by reference herein in their entirety) through feedback inhibition from the TRN (Crabtree, et al., “New Intrathalamic Pathways Allowing Modality-Related and Cross Modality Switching in the Dorsal Thalamus.” J. Neurosci. 22, 8754-8761 (2002), the disclosure of which is incorporated by reference herein in its entirety).

### BRIEF DESCRIPTION OF THE DRAWINGS

FIG. 1 is a block diagram of an exemplary system of the present technology for vector-based targeting of a human central thalamus to guide deep brain stimulation including a surgical computing device.

FIG. 2 is a partial side view and partial block diagram of an exemplary deep brain stimulation apparatus of the present technology.

FIG. 3A is a partial side view and partial block diagram of one embodiment of a deep brain stimulation apparatus of the present technology implanted in a brain.

FIG. 3B is a perspective view of a portion of the deep brain stimulation apparatus implanted as shown in FIG. 3A to activate central thalamus fibers in a subject.

FIG. 4 is a block diagram of the adaptive feedback controller illustrated in FIG. 3A.

FIG. 5 is a flowchart of an exemplary method for surgical planning involving vector-based targeting of a human central thalamus to guide deep brain stimulation.

FIG. 6 illustrates methods used for image-guided surgical planning to facilitate vector-based targeting of a human central thalamus to guide deep brain stimulation.

FIG. 7 illustrates white matter null (WMn) imaging showing contrast within a thalamus to allow identification of individual thalamic nuclei.

FIG. 8 illustrates a combination of WMn and diffusion tensor image (DTI) imaging that provides both target and avoidance nuclei, as well as target and avoidance fiber tracts, which are used to define vector-based targeting that takes into account both the position and the trajectory (i.e., orientation) of the DBS leads (e.g., electrode contacts) relative to the target projections from the nucleus and the fiber bundles emanating from this nucleus.

FIGS. 9A and 9B illustrate a conceptual overview showing placement of a vector in a three-dimensional collection of fibers to be adjusted for bulk activation of fibers of the CL/DTTm structure.

FIG. 10 illustrates a volumetric rendering of two thalamic nuclei (activation target) and centromedian (avoidance target), target DTTm fiber bundle, and a DBS lead with active electrodes.

FIG. 11 illustrates another volumetric rendering of the two thalamic nuclei of FIG. 10 with isolation of fibers activated by applied electric field.

FIG. 12 illustrates multiple target activation (CL, PPN) and avoidance pathways (MD, VPM, CM) within the human central thalamus.

FIG. 13 illustrates fiber activation profiles including histograms of percentage activation of target activation and target avoidance regions for a generic thalamic model system.

FIG. 14 illustrates changes in fiber activation achieved with adjustment of electrode position from that illustrated in FIG. 13.

FIG. 15 illustrates human thalamic imaging data from a human subject with traumatic brain injury (TBI) including the percentage activation of CL and PPN targets and other thalamic nuclei for avoidance (VPM, CM, MD).

FIG. 16 illustrates testing results for five subjects receiving DBS according to the vector-based targeting of the human central thalamus of FIG. 5.

FIG. 17 illustrates an exemplary approach to target acquisition from a representative human subject along with activation results from both hemispheres.

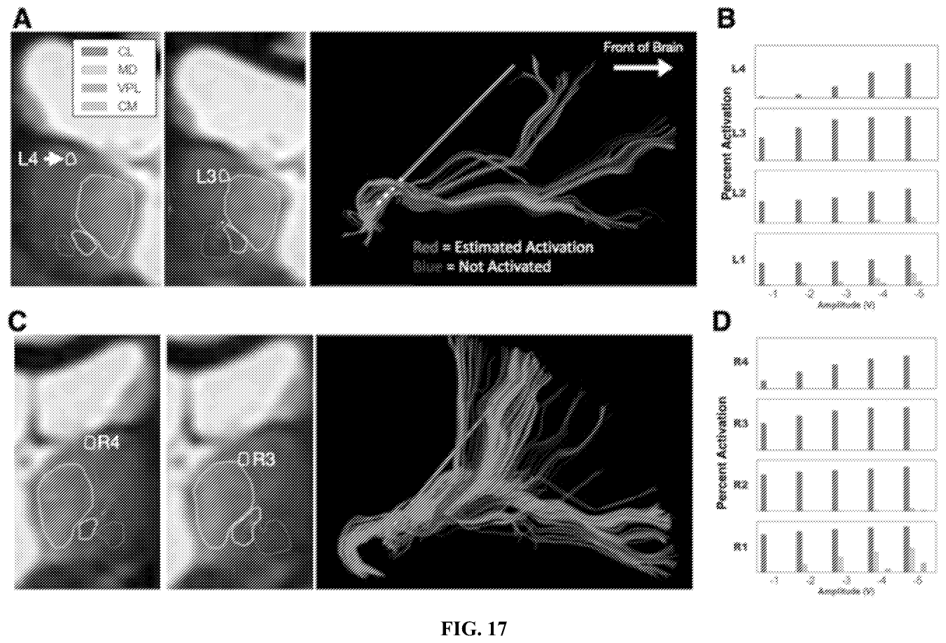

FIG. 18 illustrates another exemplary approach to target acquisition from another representative human subject along with activation results from both hemispheres.

FIG. 19 illustrates the placements of active contacts for a plurality of human subject in a common synthetic atlas space.

FIG. 20 illustrates cortical evoked potentials obtained across a 128 channel EEG array for activation across two active contacts using a 2 Hz duty cycle of stimulation

### DETAILED DESCRIPTION

The present technology relates to methods for vector-based targeting of a human central thalamus to guide deep brain stimulation (DBS). The present technology also relates to methods, devices, systems, and non-transitory computer readable media for surgical planning for vector-based targeting of a human central thalamus to guide DBS. More specifically, the present technology relates to methods of human central thalamic targeting to achieve target activation and successful target avoidance of regions of human intra-thalamic pathways within the central thalamus to achieve vector-based placement of deep brain stimulation electrodes.

Devices and systems for carrying out vector-based targeting of a human central thalamus to guide DBS, including a surgical computing device, are described herein. One aspect of the present technology relates to a system for vector-based targeting of a human central thalamus to guide DBS. The system includes a surgical computing device of the present technology. The system also includes an imaging device operationally coupled to the surgical computing device and one or more electrodes. An electrical stimulator is coupled to the surgical computing device and the one or more electrodes to permit electrical activation of the electrodes based on instructions from the surgical computing device.

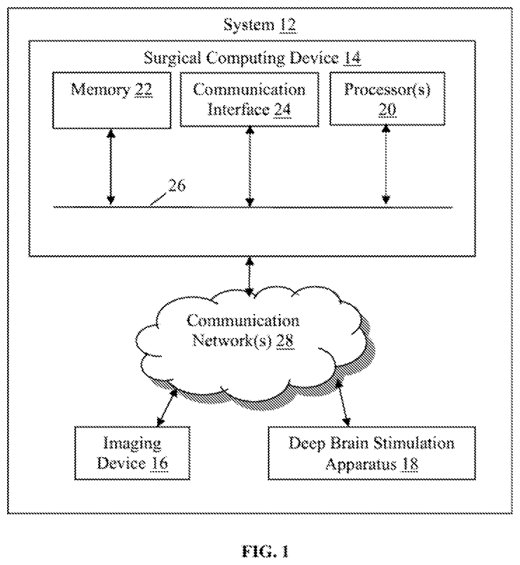

FIG. 1 illustrates an environment including system 12 for vector-based targeting of a human central thalamus to guide DBS. System 12 includes surgical computing device 14, imaging device 16, and DBS apparatus 18, although system 12 may include other elements or components in other combinations, such as additional computing devices. System 12 enables treatment via the selective activation of structures within the central thalamus to support forebrain arousal regulation via the activation of fibers emanating from the central lateral nucleus of the central thalamus (CL) and the surrounding dorsal tegmental track medial (DTTm) (CL/DTTm).

Surgical computing device 14 of system 12 includes processor(s) 20, memory 22, and communication interface 24 that are coupled together by a bus 26 or other communication link, although surgical computing device 14 can include other types and/or numbers of elements in other configurations. Processor(s) 20 of surgical computing device 14 may execute programmed instructions stored in memory 22 for any number of the functions or other operations illustrated and described by way of the examples herein, including surgical planning for vector-based targeting of a human central thalamus to guide DBS. Processor(s) 20 of surgical computing device 14 may include one or more graphic processing units (GPUs), central processing units (CPUs), or general purpose processors with one or more processing cores, for example, although other types of processor(s) can also be used.

Memory 22 of surgical computing device 14 stores these programmed instructions for one or more aspects of the present technology as illustrated and described herein, although some or all of the programmed instructions could be stored elsewhere. A variety of different types of memory storage devices, such as random access memory (RAM), read only memory (ROM), solid state drives (SSD), flash memory, or other computer readable medium that is read from and written to by a magnetic, optical, or other reading and writing system that is coupled to processor(s) 20 can be used for memory 22.

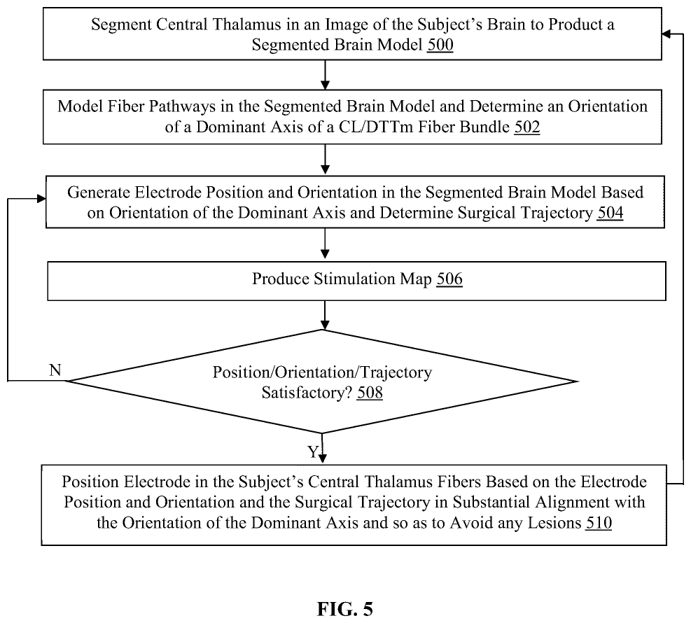

Accordingly, memory 22 of surgical computing device 14 can store application(s) that can include executable instructions that, when executed by surgical computing device 14, cause surgical computing device 14 to perform actions, such as to perform methods for vector-based targeting of a human central thalamus to guide DBS as illustrated and described by way of the examples herein, such as in FIG. 5. The application(s) can be implemented as modules or components of other application(s). Further, the application(s) can be implemented as operating system extensions, modules, plugins, or the like.

Communication interface 24 of surgical computing device 14 operatively couples and allows for communication between surgical computing device 14, imaging device 16, and DBS apparatus 18, which are all coupled together by one or more communication network(s) 28, although other types and/or numbers of connections and/or configurations to other device and/or elements can be used. Communication network(s) 28 can include any number and/or types of communication networks, such as local area network(s) (LAN(s)) or wide area network(s) (WAN(s)), and/or wireless networks, although other types and/or number of protocols and/or communication network(s) can be used.

Although embodiments of surgical computing device 14 are described and illustrated herein, surgical computing device 14 can be implemented on any suitable computing system or computing device. It is to be understood that the devices and systems described herein are for exemplary purposes and many variations of the specific hardware and software are possible, as will be appreciated by those skilled in the relevant art(s).

In addition, two or more computing systems or devices can be substituted for any one of the systems described above. Accordingly, principles and advantages of distributed processing, such as redundancy and replication, also can be implemented, as desired, to increase the robustness and performance of the devices and systems described above. The embodiments of the present application may also be implemented on a computer system or systems that extend across any suitable network using any suitable interface mechanisms and communications technologies, including, by way of example only, telecommunications in any suitable form (e.g., voice and modem), wireless communications media, wireless communications networks, cellular communications networks, G3 communications networks, Public Switched Telephone Networks (PSTNs), Packet Data Networks (PDNs), the Internet, intranets, and combinations thereof.

Imaging device 16 may be any suitable imaging device to obtain images of the subject's brain, including devices suitable for computed tomography imaging, although other appropriate imaging devices may be employed. Imaging device 16 is coupled to surgical computing device 14 to provide images of the subject's brain for further analysis in accordance with the methods disclosed herein.

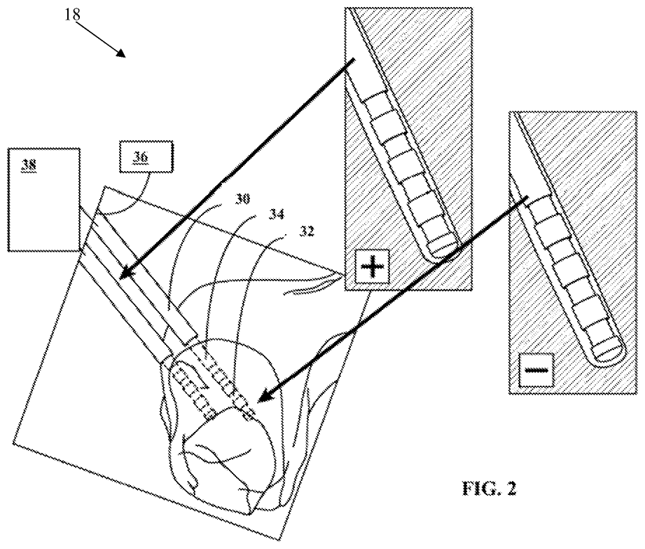

FIG. 2 is a perspective view and functional block diagram of DBS apparatus 18. DBS apparatus 18 includes first and second stimulators 30 coupled to stimulus signal generator 32. Although DBS apparatus 18 is described with respect to first and second stimulators 30, it is to be understood that DBS apparatus 18 may include additional stimulators. Further, although DBS apparatus 18 is described, it is to be understood that other types of stimulation devices could be employed in the methods of the present technology including stimulation devices that employ other energy modalities.

First and second stimulators 30 include at least one electrode 32 mounted on shank 34. In one embodiment, more than one electrode 32 is mounted on shank 34 such that stimulator 30 is a “multipolar electrode,” with each electrode separately controllable. In this example, four electrodes 32 are located on each shank 34 to provide a plurality of spaced contacts, although other numbers of electrodes may be utilized. Electrodes 34 are connected to one (or separate) insulated conductor(s) which passes through shank 34. The insulated conductor connects electrodes 32 to voltage control 36 and stimulus signal generator 38. Voltage control 36 and stimulus signal generator 38 may be separate from one another or part of a single unit. The connections mentioned herein may be wired or wireless.

Electrodes 32 are made from a conducting material, which may be an alloy such as platinum/iridium, with impedances known in the art, for example, between approximately of 100 and 150 kΩ. Electrodes 32 are approximately 0.5 mm in length. In one embodiment, where multiple electrodes 32 are mounted on shank 34, the separation between electrodes 32 may be variable or constant, and may be approximately 0.5 mm.

Shank 34 is configured to be implanted in the brain of the subject. Shank 34 may be configured as a cylinder, a square, a helix, or any other geometry known in the art as suitable for implementation. In one embodiment, shank 34 is implanted in the central thalamus of the subject for selective activation of central thalamus fibers in the subject, as described herein.

Stimulus signal generator 38 produces a selected pulse train. In one embodiment, stimulus signal generator 38 is capable of separately driving individual electrodes 32 in a multi-electrode system through various channels. In this example, stimulus signal generator 38 may operatively select any one of electrodes 32 to provide a stimulus signal. Stimulus signal generator 38 may provide stimulation with various parameters, such as frequency or waveform, across multiple electrodes 32 simultaneously, and independently.

Stimulus signal generator 38 is capable of generating voltage wave trains of any desired form (monophasic or biphasic sine, square wave, spike, rectangular, triangular, ramp, etc.) in a selectable voltage amplitude in the range from about 0.1 volts to about 10.5 volts or from about 0.1 mA to about 25.0 mA and at selectable frequencies in the range from about 1 Hz to about 10 kHz. In one embodiment, stimulus signal generator 38 is capable of generating constant current across at least one pair of electrodes 30 with either electrode in the pair assigned as a cathode or anode, although stimulus signal generator 38 may generate a constant current across two pairs of electrodes, across four pairs of electrodes, or across six pairs of electrodes, where either electrode in a pair can be assigned as a cathode or an anode. The compliance voltage of stimulus signal generator 38 is able to handle resistive loads across any pair of electrodes in the range from 0.5 kOhm to 10 kOhm. Each channel (cathode/anode pair) is able to deliver up to about 25.0 mA.

Stimulus signal generator 38 includes circuitry that allows for monitoring of the current delivered across each channel. In one embodiment, stimulus signal generator 38 is programmable in that pulse shapes, sequences, and frequencies of pulses can be designed by software on a computer, such as surgical computing device 14, and uploaded to stimulus signal generator 38 for delivery to electrodes 32 upon command. The cathode-anode outputs from each channel may be used to provide bipolar constant-current stimulation in the intralaminar nuclei through any pair of electrode contacts across implanted stimulators 30.

Voltage control 36 provides a selected current amplitude or voltage to the waves of the pulse train. In practice, the pulse train and voltage amplitudes employed will be selected on a trial and error basis by evaluating a subject's response to various types and amplitudes of electrical stimulation over a time course of from about 1 to about 12 months. For example, after implanting stimulators 30 in the subject's thalamic nuclei, stimulation with a voltage within the range of from about 0.1 to about 10.5 volts or higher at a rate within the range of from about 1 Hz to about 10 kHz, is applied for from about 8 to about 12 hours a day. The voltage control 36 may provide continuous, periodic, or intermittent stimulation. In one embodiment, voltage control 36 provides an electrical stimulus that is carried out using one or more stimulation programs that are capable of being interleaved in time.

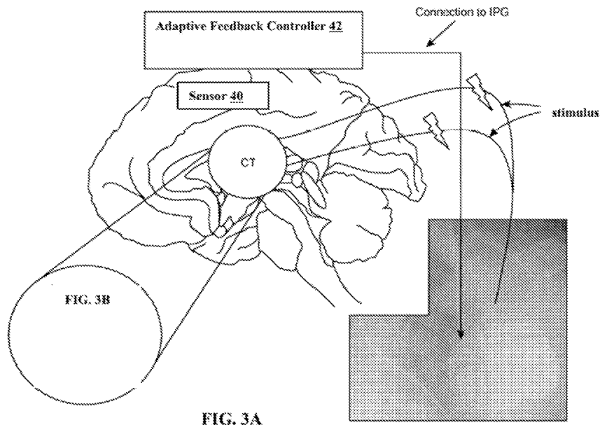

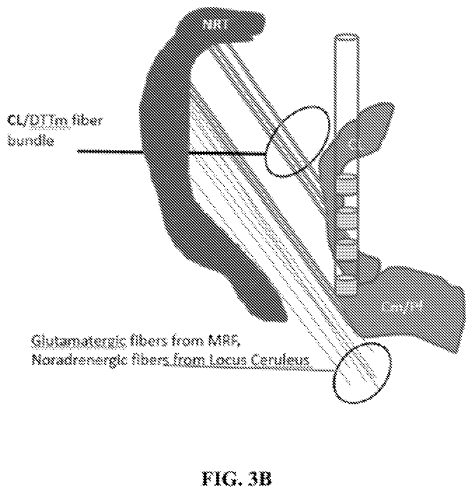

Referring now to FIGS. 3A and 3B, in one embodiment, DBS apparatus 18 includes one or more sensors 40 connected to adaptive feedback controller 42. Sensors 40 are configured to detect neuronal activity of one or more cortical and/or subcortical tissues of a selected subject's brain, by means known in the art, although electrodes 32 may be utilized to detect neuronal activity. In one embodiment, sensors 40 are incorporated into stimulators 30, although sensors 40 not incorporated into a stimulator, referred to herein as “extra-stimulator sensors” may be utilized. The extra-stimulator sensors may be implanted within cortical or subcortical regions or may be located on the scalp surface of the subject's head. Sensors 40 collect neuronal data in the form of, for example, single-unit activity, local field potentials, and/or electrocorticogram (“EcoG”) activity. Connections between sensor 40 and brain tissue may be electrical, electromagnetic (wireless), or optical to one or many targets to be determined by availability and involvement in specific patterns of brain injury.

In one embodiment, sensors 40 include computer and logic circuitry, although computer and logic circuitry associated with sensors 40 may be distributed among other components, such as incorporated into adaptive feedback controller 42, or in the stimulus signal generator 38, and/or one or more other devices, which may be implanted in the subject or external to the subject. In one embodiment, cortical placement of sensors 40 can detect the occurrence of failures of human control and adaptive feedback 42 controller can adjust stimulation of thalamic targets in synchronism with the processes occurring in deep brain stimulation apparatus 18.

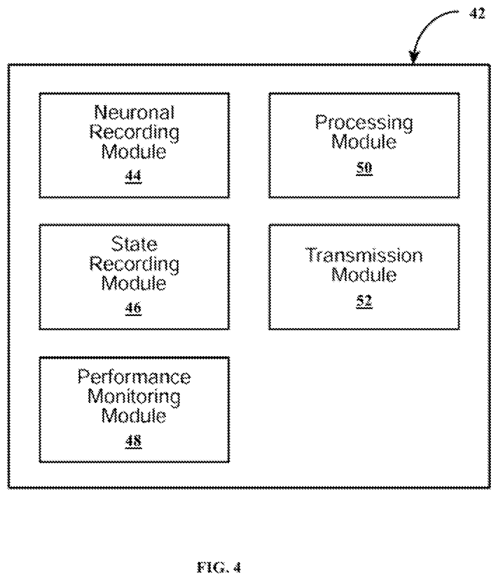

Referring now to FIGS. 3A, 3B, and 4, in one embodiment, adaptive feedback controller 42 includes neuronal recording module 44, state monitoring module 46, performance monitoring module 48, processing module 50, and transmission module 52. The modules described here for adaptive feedback controller 42 may be located within one physical device or may be distributed among multiple devices, including surgical computing device 14, and may be incorporated with other components or devices described herein. For example and without limitation, neuronal recording module 44 may be located in the same device as an extra-stimulator sensor and said device will have appropriate transmission pathways to receive and send information from and to other components of DBS apparatus 18, the subject, and/or external systems used to maintain, control, or inspect deep brain stimulation apparatus 18 or the subject, including surgical computing device 14.

Neuronal recording module 44 receives and stores various items of information from sensors 40, such as electrical waveform pattern data unique to the subject. In one embodiment, neuronal recording module 44 stores information received from sensors 40 in real-time when DBS apparatus 18 is being used. In one embodiment, neuronal recording module 44 includes output means to allow retrieval of signals stored during an off-line operation of DBS apparatus 18.

State monitoring module 46 is coupled to sensors 40, and is configured to store and process a first set of variables associated with a state of the detected neuronal activity, particularly the spectral content of the local neuronal activity and in particular, the total power within the frequency ranges 10-15 Hz, 15-20 Hz, 20-25 Hz, 25-30 Hz, 10-30 Hz, which have all been empirically identified to increase within neuronal populations of the cortex, basal ganglia, and thalamus during either effective multi-site stimulation or during alert cognitive function. State monitoring module 46 may be used to sample the average characteristics of neuronal activity over time from sensors 40 or outside of the brain that collect neuronal signals for this purpose and to provide as feedback the real-time characteristics of the signals via direct or wireless (Bluetooth) connections. In one embodiment, state monitoring module 46 includes an internal memory and computational resources to extract signal features of the neuronal signal.

Performance monitoring module 48 is coupled to sensors 40 and is configured to store and process a second set of variables associated with modulation of the frequency of the locally detected neuronal activity. Performance monitoring module 48 is used to monitor the performance characteristics of the stimulation in producing increases in spectral power of local populations at pre-specified frequency ranges (e.g., 15-25 Hz). In one embodiment, performance monitoring module 48 includes an internal memory and computational resources to extract signal features of the neuronal signal.

Processing module 50 is coupled to state monitoring module 46 and performance monitoring module 48. In one embodiment, processing module 50 is configured to extract a feature vector based upon the processed first and second set of variables, and may be configured to compute an optimal response stimulus signal based upon a comparison between the extracted feature vector and a pre-stored feature vector corresponding to the local spectrum of neuronal activity for the subject recording sites. Transmission module 52 is configured to transmit the optimal response stimulus signal computed by the processing module 50 to the implanted stimulus signal generator 38 to regulate the arousal level neuronal activity of the subject.

Based upon respective sets of variables stored and/or measured, performance monitoring module 48 and state monitoring module 46 may be used to extract a feature vector from the variables using computer and logic circuitry. Feature vectors represent an approximately complete mathematical description of electrical signals resulting from neuronal activity. Computed feature vectors can be used for further processing and to synthesize a feedback signal if necessary. A feedback signal can be outputted via a transmission path, which may be wired, wireless, or optical as known to one skilled in the art. The same or a separate component of DBS apparatus 18 computes an output signal and transmits it to stimulator 30 placed within the brain to regulate their output in response to ongoing analysis provided by internal monitoring systems.

Referring again to FIGS. 3A and 4, an embodiment of the present application wherein the DBS apparatus 18 includes sensors 40 that are interfaced to adaptive feedback controller 42, which in turn is interfaced to stimulus signal generator 38, is shown. Stimulus signal generator 38 is configured to provide feedback control of electrical stimulation of the targeted brain regions, for example, the CL/DTTm fiber pathways. Upon receipt of a signal via a transmission path, which may be wired, wireless, or optical, stimulus signal generator 38 provides a corresponding stimulus to these regions of the brain via at least one of stimulators 12 to modulate or maintain the arousal state of a subject. The operating characteristics of DBS apparatus 18 may be adjusted automatically using adaptive feedback controller 42. In other embodiments, sensor 40 or components of adaptive feedback controller 42 may store information for retrieval by an external system or by a physician, or may be used by a physician/programmer to adjust DBS apparatus 18 settings. Settings may be adjusted by the DBS apparatus 18 itself or by an external physician/programmer to raise a level of arousal, or impact on local signal power.

One aspect of the present technology relates to a method for vector-based targeting of a human central thalamus to guide deep brain stimulation (DBS). The method involves providing one or more electrodes each with a plurality of contacts. A three-dimensional orientation of a dominant axis of a CL/DTTm fiber bundle of the human subject is determined. The plurality of contacts of the one or more electrodes are then positioned in the human subject's central thalamus fibers in substantial alignment with the determined three-dimensional orientation of the dominant axis of the CL/DTTm fiber bundle. An electrical stimulus is then applied to the positioned plurality of contacts of the one or more electrodes to treat the human subject for impaired arousal regulation. The positioning and the applying are carried out to maximize activation of a central lateral nucleus and medial dorsal tegmental tract fiber pathway in the human subject and to minimize activation of a centromedian-parafascicularis fiber pathway in the human subject.

In a first step, one or more electrodes each with one or more contacts are provided. In one embodiment, deep brain stimulator apparatus 18 with electrodes 32 may be employed, although other devices for activation of the subject's central thalamus may employed such as a fiberoptic-optogenetic (“FOG”) system, BION system, or ultrasound. The one or more electrodes are configured to provide for selective activation of the central thalamus fibers of the subject as described below. The present technology may be employed with single lead systems with multiple electrical contacts, single lead systems with multiple split contacts, and multiple lead systems with any combination of multi-contact electrodes including split band contacts. Importantly, the system will be capable of addressing any combination of anodes and cathodes across lead(s) contacts.

Next, the one or more electrodes, such as electrodes 32 are positioned in the subject's central thalamus fibers. In one embodiment, once a relevant subject is selected, stimulator 30, as described above, is implanted in the subject's central thalamus as illustrated in FIG. 3B to maximize central lateral nucleus and medial dorsal tegmental tract fiber pathway activation in the subject and to minimize central medial parafascicularis fiber pathway activation in the subject. The zones of activation and suppression are illustrated in FIG. 5. As discussed above, stimulator 30 includes one or more electrodes 32. In some embodiments, a plurality of electrodes 32 are provided. One or more electrodes 32 have a plurality of spaced contacts. The CL/DTTm target can be activated optimally by shaping the applied electrical field by utilizing first and second stimulators 12, with many electrode 32 contacts as described below. As shown, this is achieved by positioning the most of electrodes 32 on stimulator 30 to be in contact with the central lateral nucleus and medial dorsal tegmental tract fibers while few if any of electrodes 32 on stimulator 30 are in contact with the central median parafascicularis fibers.

To carry out the above methods, a subject may be conscious with application of local anesthesia or mild sedation. In cases where a subject is not sufficiently cooperative to remain conscious during the procedure, the above-described approach can be modified in ways known in the art, to allow the operation to be completed under general anesthesia.

Subjects may include any animal, including a human. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

The methods described herein can be employed for subjects of any species, gender, age, ethnic population, or genotype. Accordingly, the term subject includes males and females, and it includes elderly, adult-to-elderly transition age subjects adults, pre-adult-to-adult transition age subjects, and pre-adults, including adolescents, children, and infants. In one embodiment, subjects are adult subjects in their twenties to forties, who have the most to gain from treatment and represent the greatest cost to society if left untreated. Examples of human ethnic populations include Caucasians, Asians, Hispanics, Africans, African Americans, Native Americans, Semites, and Pacific Islanders. The term subject also includes subjects of any genotype or phenotype as long as they are in need of the treatment as described herein. In addition, the subject can have the genotype or phenotype for any hair color, eye color, skin color or any combination thereof. The term subject includes a subject of any body height, body weight, or any organ or body part size or shape

In one embodiment, stimulator 30 is introduced via burr holes in the skull, although in other examples multiple stimulators may be employed. Generally, prior to the introduction of stimulators 30, a detailed mapping with microelectrode and microstimulation following standard methods is carried out as described in Tasker et al., “The Role of the Thalamus in Functional Neurosurgery,” Neurosurgery Clinics of North America 6 (1): 73-104 (1995), which is incorporated herein by reference in its entirety. Imaging device 16 may be employed to image the subject's brain. The system will enable the user to plan an implantation of a stimulation system, such as stimulator 30, in an individual subject using the neuroimaging data from imaging device 16.

The imaging data is employed to model thalamic nuclei, white matter fiber tracts and connections, and the impact of electrical field activation within the thalamus by directly modeling the relative activation of CL/DTTm→TRN, Cm-Pf→TRN, and other adjacent thalamic pathways. The present technology enables the biophysical modeling of the precise placement of a single or multiple lead system to selectively activate CL/DTTm and avoid co-activation of the Cm-Pf fiber bundle. This system includes modeling of thalamic nuclei, modeling of specific white matter fiber pathways within the brain, bioelectric field modeling, and probabilistic mapping of target activation and target avoidance achieved with varying configurations of lead contact arrangements, cathode and anode geometries, pulse shapes, pulse widths, and frequencies of stimulation.

In one aspect, a segmented brain model of the subject's central thalamus may be produced using known techniques. Model electrode positions and electrical stimulation conditions may be identified using the segmented brain model that will maximize central lateral nucleus and medial dorsal tegmental tract fiber pathway activation in the subject, while minimizing the central medial parafascicularis fiber pathway activation in the subject. A stimulation map is produced based on the identified electrode positions and electrical stimulation conditions. The stimulation map may then be employed to carry out the actual positioning of the system, such as stimulator 30. The stimulation map, in some examples, may also be used to determine the applying of stimulation, as described further below.

Next, an electrical stimulus is applied to the positioned one or more electrodes 32 to selectively activate the central thalamus fibers of the subject. The electrical stimulus may be carried out in various conditions to maximize central lateral nucleus and medial dorsal tegmental tract fiber pathway activation in the subject and to minimize central medial parafascicularis fiber pathway activation in the subject. For example, the electrical stimulus may be applied between 0.1 to 25.0 milliamps or 0.1 to 10.5 volts, selected independently for each electrode. The electrical stimulus may be applied using continuous, intermittent or periodic stimulation. The electrical stimulus may be applied using substantially in-phase or substantially out-of-phase stimulation on each electrode 32. The electrical stimulus can be configured to be ramped up or down at different rates of speed to improve the selective activation. The electrical stimulus is carried out using voltage wave trains having a monophasic or biphasic sine, square, spike, rectangular, triangular or ramp configurations. The electrical stimulus can be applied at one or more frequencies of from 1 Hz to 10 kHz. Further, the electrical stimulus can be carried out using one or more stimulation programs that are capable of being interleaved in time.

The devices and systems of the present technology allow for the precise placement of single or multiple leads to selectively activate CL/DTTm fibers and minimize adjacent OFF-target fibers originating and passing through the centromedian-parafasicularis nucleus complex (Cm-Pf) that also project to the thalamic reticular nucleus (TRN), such as shown in FIG. 3B. The one or more electrodes 32 are positioned to maximize central lateral nucleus and medial dorsal tegmental tract fiber pathway activation in the subject and to minimize central median parafascicularis fiber pathway activation in the subject as shown in FIG. 5.

The present technology specifies the geometric requirements for selective activation of CL/DTTm to facilitate cognitively mediated behaviors (including but not limited to executive functions, vigilance, sustained attention, working memory, decision-making, and motor executive functions (e.g. controlled hand and arm movements). The primary effect of selective CL/DTTm stimulation is activation of neuronal populations across frontal cortical structures and the striatum, while minimizing OFF-target effects. Other cortical structures such as posterior parietal cortices and primary sensory cortices are additional direct targets of CL/DTTm activation based on known anatomical and physiological demonstrations. In one embodiment, 75% to 100% of the medial dorsal tegmental tract fibers in the central thalamus of the subject are stimulated and less than 25% of the central medial parafascicularis fibers in the central thalamus of the subject are stimulated. In another embodiment, 90% to 100% of the medial dorsal tegmental tract fibers in the central thalamus of the subject are stimulated and less than 10% of the central median parafascicularis fibers in the central thalamus of the subject are stimulated.

In one embodiment, deep brain stimulation apparatus 18 further includes sensors 40 that are configured to provide feedback to determine a state of neuronal activity during application of an electrical stimulus as described above. One or more of the electrical stimulus conditions can be adjusted based on the state of neuronal activity to provide improved selective activation of the subject's central thalamus based on feedback from sensors 40.

Another aspect of the present technology relates to a method of treating a condition characterized by impaired arousal regulation in a human subject. The method involves selecting a human subject with impaired arousal regulation. One or more electrodes are provided each with a plurality of contacts. A three-dimensional orientation of a dominant axis of a CL/DTTm fiber bundle of the human subject is determined. The plurality of contacts of the one or more electrodes are then positioned in the human subject's central thalamus fibers in substantial alignment with the determined three-dimensional orientation of the dominant axis of the CL/DTTm fiber bundle. An electrical stimulus is then applied to the positioned plurality of contacts of the one or more electrodes to selectively activate the central thalamus fibers in substantial alignment with the determined three-dimensional orientation of the dominant axis of the CL/DTTm fiber bundle. An electrical stimulus is then applied to the positioned plurality of contacts of the one or more electrodes to selectively activate the central thalamus fibers of the human subject. The positioning and the applying are carried out to maximize activation of a central lateral nucleus and medial dorsal tegmental tract fiber pathway in the human subject and to minimize activation of a centromedian-parafascicularis fiber pathway in the human subject.

Impaired arousal regulation is a key underlying component of a wide range of acquired, inherited, and idiopathic neuropsychiatric illnesses. Most prominently, traumatic brain injuries produce impaired arousal regulation. Additional forms of structural brain injuries that disrupt arousal regulation include anoxia, hypoxia, hypoxic-ischemic injuries, stroke, encephalitis of infectious or autoimmune causes, and a wide range of primary degenerative illnesses such as Parkinson's disease. Importantly, supporting arousal regulation is under present clinical study for restoring cognitive function during seizures or post-ictal states of depressed cortical function. Impaired arousal regulation is an untreated primary feature of neuropsychiatric diseases such as schizophrenia or autism. Accordingly, the technology described and illustrated herein can be used to treat brain injury, a neurological degenerative disease, epilepsy, a movement disorder, a post-encephalitis cognitive impairment, a development disorder, a post-hypoxic-ischemic injury cognitive impairment, a neuropsychiatric disorder, post-intensive care unit (ICU) mixed disorder cognitive impairment, and/or post-ICU adult respiratory distress syndrome. These applications are noted as relevant examples but are not exhaustive of applications for the specific use of the system to enable selective CL/DTTm activation in an individual to improve arousal regulation.

In one embodiment, a subject having a condition characterized by impaired arousal regulation may be selected for treatment using the method described above. The subject may have a condition selected from the group consisting of brain injury, a neurological degenerative disease, epilepsy, a movement disorder, a post-encephalitis cognitive impairment, a developmental disorder, a post hypoxic-ischemic injury cognitive impairment, and a neuropsychiatric disorder.

The present technology enables the specific positioning of a system within the central thalamus to optimize behavioral facilitation achievable with improved arousal regulation. The technology guides the conceptualization and placement of the system and allows the user to explore a space of stimulation configurations and modes of activation to map a range of behavioral outcomes to the system, as described in further detail below. These maps are inherently multi-dimensional: they include effects on CL/DTTm and Cm-Pf→TRN pathways, multiple possible behavioral facilitation effects, and just as important OFF-target side effects.

Selective activation of the DTTm fiber pathway that projects through the CL nucleus, and not the Cm-Pf complex fiber projections, facilitates performance. Such selective activation can be utilized as therapeutic options for treatment of subjects suffering from impaired arousal regulation and enduring cognitive dysfunction. As disclosed in Baker, et al., “Robust Modulation of Arousal Regulation, Performance and Frontostriatal Activity Through Central Thalamic Deep Brain Stimulation in Healthy Non-Human Primates.” J. Neurophysiol. 116:2383-2404 (2016), the disclosure of which is incorporated by reference herein in its entirety, shaping the DBS electrical field within the ‘wing’ of CL resulted in robust behavioral facilitation and enhancement of frontal and striatal population activity. These findings are consistent with the behavioral and physiological effects of conventional CT-DBS in a case study in a very severely traumatic brain injury (TBI) subject, as disclosed in Schiff, et al., “Behavioural Improvements with Thalamic Stimulation After Severe Traumatic Brain Injury.” Nature. 448, 600-3 (2007), the disclosure of which is incorporated herein by reference in its entirety.

The present technology disaggregates the CL thalamus by isolating contributions from CL and DTTm from the contributions of the Cm-Pf complex. Two mechanisms may explain these behavioral results: 1) an intrathalamic inhibitory network similar to that defined in the rodent, as disclosed in Crabtree, et al., “New Intrathalamic Pathways Allowing Modality-Related and Cross Modality Switching in the Dorsal Thalamus.” J. Neurosci. 22, 8754-8761 (2002) and Crabtree, “Functional Diversity of Thalamic Reticular Subnetworks.” Front. Syst. Neurosci. s12 (2018), the disclosures of which are incorporated by reference herein in their entirety), 2) the roles the two pathways play in controlling the anterior forebrain mesocircuit, as disclosed in N. D. Schiff, “Recovery of Consciousness After Brain Injury: A Mesocircuit Hypothesis.” Trends Neurosci. 33, 1-9 (2010), the disclosure of which is incorporated by reference herein in its entirety, a system involving the thalamus, frontal cortex, and basal ganglia that regulates the overall level of activity in the anterior forebrain.

In one embodiment, the position of segmented single leads and multi-lead systems can be optimized to selectively target the cell bodies of CL and the DTTm pathway and to avoid the fiber projections emanating from Cm-Pf. The isolated activation of the DTTm pathway projecting from CL to frontostriatal targets facilitates behavioral performance. In contrast, mixed activation of the DTTm and fibers projecting from the Cm-Pf complex through the TRN either interrupts or mitigates these facilitation effects.

Although both CL and Cm-Pf have strong striatal projections, their patterns of innervations within the striatum are markedly different, both regionally and with respect to cellular elements and cell types innervated. Single fiber studies note that CL afferents make en passant synapses in TRN before fanning out broadly over the rostral striatum as disclosed in Deschenes, et al., “Striatal and Cortical Projections of Single Neurons From the Central Lateral Thalamic Nucleus in the Rat.” Neuroscience. 72, 679-687 (1996), the disclosure of which is incorporated by reference herein in its entirety. By contrast, Cm-Pf fibers project heavily into regionally precise zones of the striatum and form bushy local arborizations, as disclosed in Parent, et al., “Axonal Collateralization in Primate Basal Ganglia and Related Thalamic Nuclei.” Thalamus Relat. Syst. 2, 71 (2002), Smith, et al., “The Thalamostriatal Systems: Anatomical and Functional Organization in Normal and Parkinsonian States.” Brain Res. Bull. 78, 60-68 (2009), Storch, et al., “Reliability and Validity of the Yale Global Tic Severity Scale.” Psychol. Assess. 17, 486-491 (2005), and Smith, et al., “The Thalamostriatal System in Normal and Diseased States.” Front. Syst. Neurosci. 8 (2014), the disclosures of which are incorporated by reference herein in their entirety. CL and Pf afferents are known to project into the main neuronal populations of the striatum, the medium spiny neurons, as disclosed in (Bolam, et al., “Synaptic Organisation of the Basal Ganglia.” J. Anat. 196, 527-542 (2000) and Ellender, et al., “Heterogeneous Properties of Central Lateral and Parafascicular Thalamic Synapses in the Striatum.” J. Physiol. 591, 257-72 (2013), the disclosures of which are incorporated by reference herein in their entirety), whereas Cm neurons project into the local cholinergic inhibitory neurons, as disclosed in Smith, et al., “The Thalamostriatal Systems: Anatomical and Functional Organization in Normal and Parkinsonian States.” Brain Res. Bull. 78, 60-68 (2009), the disclosure of which is incorporated herein by reference in its entirety. Most importantly, CL fibers have strong and broad frontostriatal projections that strongly activate the entire frontal/prefrontal cortex and rostral striatum with high-frequency stimulation, as disclosed in Li et al., “Uncovering the Modulatory Interactions of Brain Networks in Cognition with Central Thalamic Deep Brain Stimulation Using Functional Magnetic Resonance Imaging.” Neuroscience. 440, 65-84 (2020), Liu, et al., “Frequency-Selective Control of Cortical and Subcortical Networks by Central Thalamus.” Elife. 4, 1-27 (2015), and Baker, et al., “Robust Modulation of Arousal Regulation, Performance and Frontostriatal Activity Through Central Thalamic Deep Brain Stimulation in Healthy Non-Human Primates.” J. Neurophysiol. 116:2383-2404 (2016), the disclosures of which are incorporated by reference herein in their entirety.

Despite these distinctions, improved arousal and facilitation of behavior have been reported for electrical stimulation of both CL and Cm-Pf. In rodent studies, electrical stimulation of CL facilitates object recognition memory (Shirvalkar, et al., “Cognitive Enhancement with Central Thalamic Electrical Stimulation.” Proc. Natl. Acad. Sci. U.S.A 103, 17007-17012 (2006), the disclosure of which is incorporated by reference herein in its entirety), working memory (Chang, et al., “Modulation of Theta-Band Local Field Potential Oscillations Across Brain Networks With Central Thalamic Deep Brain Stimulation to Enhance Spatial Working Memory.” Front. Neurosci. 13 (2019), the disclosure of which is incorporated by reference herein in its entirety), and decision-making (Mair, et al., “Memory Enhancement with Event-Related Stimulation of the Rostral Intralaminar Thalamic Nuclei.” J. Neurosci. 28, 14293-14300 (2008) and Mair, et al., “Cognitive Activation by Central Thalamic Stimulation: The Yerkes-Dodson Law Revisited.” Dose-Response. 9, 313-331 (2011), the disclosures of which are incorporated by reference herein in their entirety). In healthy NHPs, CL dominant stimulation, that includes the DTTm as shown here, facilitates sustained attention, working memory, and pattern-recognition behaviors as disclosed in Baker, et al., “Robust Modulation of Arousal Regulation, Performance and Frontostriatal Activity Through Central Thalamic Deep Brain Stimulation in Healthy Non-Human Primates.” J. Neurophysiol. 116:2383-2404 (2016), the disclosure of which is incorporated by reference herein in its entirety. In humans, CL stimulation has shown facilitation of a range of cognitive behaviors including motor executive function and speech production, as disclosed in Schiff, et al., “Behavioural Improvements with Thalamic Stimulation After Severe Traumatic Brain Injury.” Nature. 448, 600-3 (2007), the disclosure of which is incorporated herein by reference in its entirety. However, human studies also report speech facilitation with Cm-Pf stimulation (Bhatnagar, et al., “Effects of Intralaminar Thalamic Stimulation on Language Functions.” Brain Lang. 92, 1-11 (2005), the disclosure of which is incorporated by reference herein in its entirety) and restoration of arousal in severe brain injury.

In rodents, Crabtree, et al., “New Intrathalamic Pathways Allowing Modality-Related and Cross Modality Switching in the Dorsal Thalamus.” J. Neurosci. 22, 8754-8761 (2002), the disclosure of which is incorporated by reference herein in its entirety, demonstrated a structural basis for a rich system of intrathalamic inhibitory interactions and characterized two important findings relevant to the present results: 1) a rich network exists for local inhibition within the thalamus of separate sensory nuclei or motor nuclei; these inhibitory networks appear to be local to either sensory or motor thalamic nuclei; and 2) a cross sensory-to-motor thalamus pathway via the inhibition of the anterior intralaminar group by the caudal intralaminar group. Activation of the caudal intralaminar group produced powerful inhibition and suppression of neuronal firing in the anterior group via a disynaptic connection with TRN. These findings suggest an important motif of intra-thalamic inhibition of the two intralaminar nuclear groups in the thalamus. However, an important distinction in the rodent compared with feline or primate thalamus is the inclusion by Crabtree and Issac of CL as part of the caudal intralaminar group, in large part because the Cm-Pf nucleus is not present in the rodent as disclosed in Jones, The Thalamus Springer US, Boston, MA, ed. 2nd, 2007, the disclosure of which is incorporated by reference here in its entirety.

In comparison, Cm-Pf in primates is massively expanded (Jones, et al., “Differential Calcium Binding Protein Immunoreactivity Distinguishes Classes of Relay Neurons in Monkey Thalamic Nuclei.” Eur. J. Neurosci. 1, 222-246 (1989) and Jones, The Thalamus Springer US, Boston, MA, ed. 2nd, 2007, the disclosures of which are incorporated by reference here in their entirety), and CL has been classified as a component of the rostral intralaminar group. Jones, The Thalamus Springer US, Boston, MA, ed. 2nd, 2007, the disclosure of which is incorporated by reference here in its entirety, particularly notes that the paralamellar MD densocellular components can be considered posterior cells of the CL nucleus; these neurons strongly project to frontal and pre-frontal cortices and are contiguous with medial aspects of Cm-Pf and the anterior aspects of Pf. Several anatomists have argued for these regions to be included in the human CL nucleus, as disclosed in Jones, The Thalamus Springer US, Boston, MA, ed. 2nd, 2007, the disclosure of which is incorporated by reference here in its entirety. Detailed studies of Cm-Pf and CL interactions through the TRN are not available in non-human primate, and current modeling can be guided only by the observations in the rodent. A direct inhibitory effect on CL and surrounding association nuclei through TRN projections activated by the Cm-Pf-TRN fiber bundle can explain the apparent interference when activation is balanced in the DTTm and Cm-Pf-TRN fibers and the mitigation of this interference, with a ‘push-pull’ effect tipping toward behavioral release as the DTTm becomes relatively more engaged.

DTTm activation facilitates selective activation of frontostriatal neurons in the awake state. Prior studies have demonstrated that facilitation of cognitively mediated behaviors in the healthy NHP requires a sufficiently powerful activation of frontal and striatal neurons to alter local field potential, as disclosed in Baker, et al., “Robust Modulation of Arousal Regulation, Performance and Frontostriatal Activity Through Central Thalamic Deep Brain Stimulation in Healthy Non-Human Primates.” J. Neurophysiol. 116:2383-2404 (2016), the disclosure of which is incorporated by reference herein in its entirety, and individual neuronal spiking dynamics. In the awake state, both frontal neocortical neurons and striatal medium spiny neurons are depolarized and receive a high rate of synaptic input, as disclosed in Steriade, et al., “Natural Waking and Sleep States: A View From Inside Neocortical Neurons.” J. Neurophysiol. 85, 1969-1985 (2001) and Grillner, et al., “Microcircuits in Action—From CPGs to Neocortex.” Trends Neurosci. 28, 525-533 (2005), the disclosures of which are incorporated by reference herein in their entirety. Thus, to create sufficient impact as to be measurable in behavioral facilitation, the effects of DBS must be both spatially broad and strongly effective across frontostriatal populations.

Stimulation of CL with microelectrode techniques in awake NHPs demonstrated modest effects of behavioral facilitation, as disclosed in Smith, et al., “The Thalamostriatal Systems: Anatomical and Functional Organization in Normal and Parkinsonian States.” Brain Res. Bull. 78, 60-68 (2009), the disclosure of which is incorporated herein by reference in its entirety. In contrast, the marked increase of behavioral facilitation achieved by effective geometries produced by ‘field-shaping’ within the central thalamus (fsCT-DBS) when directly compared with conventional CT-DBS, can be first understood in the context of bulk activation across frontostriatal networks, as disclosed in Baker, et al., “Robust Modulation of Arousal Regulation, Performance and Frontostriatal Activity Through Central Thalamic Deep Brain Stimulation in Healthy Non-Human Primates.” J. Neurophysiol. 116:2383-2404 (2016), the disclosure of which is incorporated by reference herein in its entirety. In human subjects, bulk activation of frontostriatal neuronal populations has been demonstrated as a common mechanism underlying a variety of effective pharmacological and electrophysiological stimulation treatment methods aimed at improving arousal regulation in the injured brain.

In rodents, optogenetic stimulation of local neuronal populations within the central thalamus demonstrates that CL stimulation uniquely activates the entire frontostriatal system as measured at the whole brain level using functional magnetic resonance, as disclosed in Liu, et al., “Frequency-Selective Control of Cortical and Subcortical Networks by Central Thalamus. Elife. 4, 1-27 (2015), the disclosure of which is incorporated herein by reference in its entirety. The selective effect of stimulation of DTTm fibers demonstrated here is consistent with CL stimulation providing a broad excitatory input across frontal cortical and striatal regions. Even limited co-activation of the Cm-Pf→TRN fibers had a suppressive effect on behavior draws attention to the further distinctions of CL neurons and those within the parafascularis (Pf) and centromedian (Cm) nuclei.

The distinctions between CL and Cm-Pf neurons extend to their postsynaptic effects on inhibitory medium spiny neurons (MSNs), the neurons that project out of the striatum to the Globus pallidus (internal division). Whole-cell patch-clamp studies of MSNs optogenetically activated by either CL or Pf afferents show that CL afferents act through AMPA receptors and are more effective in driving MSN action potentials. Additionally, the Pf afferents, which act via NMDA receptors, generate long-term depression through mechanisms of synaptic plasticity, as disclosed in Ellender, et al., “Heterogeneous Properties of Central Lateral and Parafascicular Thalamic Synapses in the Striatum.” J. Physiol. 591, 257-72 (2013), the disclosure of which is incorporated by reference herein in its entirety. These physiological distinctions likely provide additional contributions to the mitigation of behavioral facilitation achieved through DTTm activation when Cm-Pf fibers are co-activated because these projections continue in the striatum to MSNs. The excitation of MSNs by CL leads to disynaptic disinhibition of the thalamus through the anterior forebrain mesocircuit, as disclosed in Fridman, et al., “Neuromodulation of the Conscious State Following Severe Brain Injuries.” Curr. Opin. Neurobiol. 29, 172-177 (2014), the disclosure of which is incorporated by reference herein in its entirety and Schiff, “Recovery of Consciousness After Brain Injury: A Mesocircuit Hypothesis.” Trends Neurosci. 33, 1-9 (2010), the disclosure of which is incorporated by reference herein in its entirety, and co-activation of Pf fibers can oppose this thalamic disinhibition through suppression of the MSNs. Thus, the balance between CL/DTTm and CM-Pf afferents to the MSNs becomes a means by which the overall activity level of the thalamus can be regulated.

Important distinctions at the cortical level are also expected to influence the impact of CL versus Cm-Pf activations; whereas CL innervates the cortex broadly, Cm-Pf projections are comparatively sparse, as disclosed in Jones, The Thalamus Springer US, Boston, MA, ed. 2nd, 2007, the disclosure of which is incorporated by reference here in its entirety. Within the neocortex, the broad innervation of supragranular and infragranular layers by CL afferents is associated with supralinear summation of effects across cortical columns, as disclosed in Llinás, et al., “Temporal Binding Via Cortical Coincidence Detection of Specific and Nonspecific Thalamocortical Inputs: A Voltage-Dependent Dye-Imaging Study in Mouse Brain Slices.” Proc. Natl. Acad. Sci. U. S. 816 A. 99, 449-454 (2002), the disclosure of which is incorporated herein by reference in its entirety. Collectively, it is likely that the encroachment of activation on Cm-Pf reduces the bulk activation of frontal cortical and striatal regions through local synaptic effects within the striatum where short-term depression may affect patchy regions of striatum innervated by Cm-Pf projections and interfere with behavioral facilitation, as disclosed in (Smith, et al., “The Thalamostriatal Systems: Anatomical and Functional Organization in Normal and Parkinsonian States.” Brain Res. Bull. 78, 60-68 (2009) and Ellender, et al., “Heterogeneous Properties of Central Lateral and Parafascicular Thalamic Synapses in the Striatum.” J. Physiol. 591, 257-72 (2013), the disclosures of which are incorporated by reference herein in their entirety). Additionally, powerful inhibition of cell bodies within parts of CL or paralaminar thalamic regions (that contain neurons with identical properties (Jones, The Thalamus Springer US, Boston, MA, ed. 2nd, 2007 and Münkle, et al., “The Distribution of Calbindin, Calretinin and Parvalbumin Immunoreactivity in the Human Thalamus.” J. Chem. Neuroanat. 19, 155-173 (2000), the disclosures of which are incorporated by reference herein in their entirety) via feedback inhibition from the TRN (Crabtree, et al., “New Intrathalamic Pathways Allowing Modality-Related and Cross Modality Switching in the Dorsal Thalamus.” J. Neurosci. 22, 8754-8761 (2002), the disclosure of which is incorporated by reference herein in its entirety) as described above may suppress thalamic output not captured by direct electrical stimulation.

In comparison to the broad bulk activation required to produce behavioral facilitation with CT-DBS in DTTm, recent work in anesthetized NHPs has demonstrated that very local stimulation within the CL nucleus using multiple 25 μm contacts spaced 200 μm apart could produce arousal from Propofol and isoflurane anesthesia, as disclosed in Redinbaugh, et al., “Thalamus Modulates Consciousness via Layer-Specific Control of Cortex.” Neuron, 1-10 (2020), the disclosure of which is incorporated by reference herein in its entirety. The effective electrotonic length of these microprobe contacts, which determines the current flow achieved locally, as disclosed in Ranck, “Which Elements are Excited in Electrical Stimulation of Mammalian Central Nervous System: A Review.” Brain Res. 98, 417-440 (1975), the disclosure of which is incorporated by reference herein in its entirety, is very short compared to the broad region activated by the fsCT-DBS configurations studied here. Of note, stimulation at 50 Hz but not 200 Hz was effective in producing arousal during anesthesia. In comparison, in the awake monkeys studied, stimulation at 150 Hz-225 Hz demonstrated the strongest behavioral facilitation and robust activation in frontal and striatal regions, as reflected by a marked increase in the beta and gamma frequency range and a decrease in the lower frequency bands measured directly in these locations, as disclosed in Baker, et al., “Robust Modulation of Arousal Regulation, Performance and Frontostriatal Activity Through Central Thalamic Deep Brain Stimulation in Healthy Non-Human Primates.” J. Neurophysiol. 116:2383-2404 (2016), the disclosure of which is incorporated by reference herein in its entirety. These differences likely reflect the need, in addition to achieving broad activation in the awake state, to increase levels of background synaptic activity received by neocortical and striatal neurons past particular thresholds, as disclosed in Larkum, et al., “Calcium Electrogenesis in Distal Apical Dendrites of Layer 5 Pyramidal Cells at a Critical Frequency of Back-Propagating Action Potentials.” Proc. Natl. Acad. Sci. U.S.A. 96, 14600-14604 (1999), Larkum, et al., “Dendritic Spikes in Apical Dendrites of Neocortical Layer ⅔ Pyramidal Neurons. J. Neurosci. 27, 8999-9008 (2007), and Larkum, et al., “Synaptic Integration in Tuft Dendrites of Layer 5 Pyramidal Neurons: A New Unifying Principle.” Science 325, 756-760 (2009), the disclosures of which are incorporated by reference herein in their entirety. Intrinsic integrative properties of individual neocortical neurons change with increasing levels of background synaptic input, as disclosed in Bernander, et al., “Synaptic Background Activity Influences Spatiotemporal Integration in Single Pyramidal Cells.” Proc. Natl. Acad. Sci. U.S.A. 88, 11569-11573 (1991), the disclosure of which is incorporated by reference herein in its entirety. In order to trigger dendritic electrogenesis in neocortical neurons, across all layers, incoming excitatory inputs must have frequencies higher than ˜130 Hz, as disclosed in Larkum, et al., “Calcium Electrogenesis in Distal Apical Dendrites of Layer 5 Pyramidal Cells at a Critical Frequency of Back-Propagating Action Potentials.” Proc. Natl. Acad. Sci. U.S.A. 96, 14600-14604 (1999), Larkum, et al., “Dendritic Spikes in Apical Dendrites of Neocortical Layer ⅔ Pyramidal Neurons. J. Neurosci. 27, 8999-9008 (2007), and Larkum, et al., “Synaptic Integration in Tuft Dendrites of Layer 5 Pyramidal Neurons: A New Unifying Principle.” Science 325, 756-760 (2009), the disclosures of which are incorporated by reference herein in their entirety. Similarly, the primary output neurons of the striatum, medium spiny neurons, require very high rates of background synaptic inputs to maintain membrane depolarization sufficient to generate action potentials, as disclosed in Grillner, et al., “Mechanisms for Selection of Basic Motor Programs—Roles for the Striatum and Pallidum.” Trends Neurosci. 28, 364-370 (2005), the disclosure of which is incorporated by reference herein in its entirety. Both mechanisms likely play a role in the requirement for high-frequency stimulation in the awake state, as disclosed in Schiff, “Central Lateral Thalamic Nucleus Stimulation Awakens Cortex via Modulation of Cross-Regional, Laminar-Specific Activity during General Anesthesia.” Neuron. 106, 1-3 (2020), the disclosure of which is incorporated by reference herein in its entirety.

The selective effect of 50 Hz CL stimulation in the anesthetized monkey may alternatively reflect antidromic activation of brainstem cholinergic and/or noradrenergic fibers that innervate CL. The brainstem neurons projecting to CL are known to have resonant properties at ˜40-50 Hz whereas higher frequencies of stimulation actually block action potentials, as disclosed in Garcia-Rill, et al., “Coherence and Frequency in the Reticular Activating System (RAS).” Sleep Med. Rev. 17, 227-238 (2013) and Garcia-Rill, J, et al., “The physiology of the pedunculopontine nucleus: implications for deep brain stimulation.” J. Neural Transm. 122, 225-235 (2015), the disclosures of which are incorporated by reference herein in their entirety, perhaps accounting for why others saw no effect during high-frequency stimulation.

A further aspect of the present technology relates to a method for surgical planning involving vector-based targeting of a human central thalamus to guide DBS implemented by one or more surgical computing devices. The method involves segmenting the central thalamus in an image of a bran of the human subject to produce a segmented brain model. One or more fiber pathways in the segmented brain model are modeled. A three-dimensional orientation of a dominant axis of a CL/DTTm fiber bundle of the human subject is determined based on the modelling. Initial model positions and orientations in the segmented brain model are generated for one or more electrodes based at least in part on the determined three-dimensional orientation of the dominant axis of the CL/DTTm fiber bundle of the human subject. A stimulation map is produced based on the modelling and the generating. A position and orientation for a plurality of contacts of the one or more electrodes in the human subject's central thalamus fibers and electrical stimulus conditions for the positioned and oriented plurality of contacts of the one or more electrodes are identified to selectively activate the central thalamus fibers of the human subject. This permits activation of a central lateral nucleus and medial dorsal tegmental tract fiber pathway in the human subject is maximized and activation of a centromedian-parafascicularis fiber pathway in the human subject is minimized based on the produced simulation map.

Yet another aspect of the present technology relates to a non-transitory computer readable medium having stored thereon instructions for surgical planning involving vector-based targeting of a human central thalamus to guide DBS. The non-transitory computer readable medium includes executable code that, when executed by one or more processors, causes the one or more processors to segment the central thalamus in an image of the human subject's brain to produce a segmented brain model. One or more fiber pathways in the segmented brain model are modeled. A three-dimensional orientation of a dominant axis of a CL/DTTm fiber bundle of the human subject is determined based on the modelling. Initial model positions and orientations in the segmented brain model are generated for one or more electrodes based at least in part on the determined three-dimensional orientation of the dominant axis of the CL/DTTm fiber bundle of the human subject. A stimulation map is produced based on the modelling and the generating. A position and orientation for a plurality of contacts of the one or more electrodes in the human subject's central thalamus fibers and electrical stimulus conditions for the positioned and oriented plurality of contacts of the one or more electrodes are identified to selectively activate the central thalamus fibers of the human subject so that activation of a central lateral nucleus and medial dorsal tegmental tract fiber pathway in the human subject is maximized and activation of a centromedian-parafascicularis fiber pathway in the human subject is minimized based on the produced simulation map.

Another aspect of the present technology relates to a surgical computing device. The surgical computing device includes comprising memory comprising programmed instructions stored thereon and one or more processors coupled to the memory and configured to execute the stored programmed instructions. The stored programmed instructions include segmenting the central thalamus in an image of a bran of the human subject to produce a segmented brain model. One or more fiber pathways in the segmented brain model are modeled. A three-dimensional orientation of a dominant axis of a CL/DTTm fiber bundle of the human subject is determined based on the modelling. Initial model positions and orientations in the segmented brain model are generated for one or more electrodes based at least in part on the determined three-dimensional orientation of the dominant axis of the CL/DTTm fiber bundle of the human subject. A stimulation map is produced based on the modelling and the generating. A position and orientation for a plurality of contacts of the one or more electrodes in the human subject's central thalamus fibers and electrical stimulus conditions for the positioned and oriented plurality of contacts of the one or more electrodes are identified to selectively activate the central thalamus fibers of the human subject so that activation of a central lateral nucleus and medial dorsal tegmental tract fiber pathway in the human subject is maximized and activation of a centromedian-parafascicularis fiber pathway in the human subject is minimized based on the produced simulation map.

Referring to FIG. 5, a flowchart of an exemplary method for surgical planning involving vector-based targeting of a human central thalamus to guide deep brain stimulation will now be described. The method may be performed by one or more computing devices, such as surgical computing device 14 as illustrated in FIG. 1. Referring again to FIG. 5, in step 500 the surgical computing device 14 segments the central thalamus is in image(s) of a human subject's brain to produce a segmented brain model.

In some examples, the imaging device 16 is used to acquire pre-surgical magnetic resonance imaging (MRI) image(s) of the human subject, optionally with specific features that assist with locating target activation regions and target avoidance regions. In these examples, the pre-surgical MRI image(s) include an image series that shows strong contrast between white and grey matter structures within the thalamus.

Optionally, white-matter-nulled magnetization-prepared rapid acquisition (WMnMPRAGE, or WMn) imaging of the human thalamus using MR acquisition parameters (e.g., inversion time TI, sequence repetition time TS, flip angle FA, receive bandwidth RBW, and/or k-space ordering strategy) can be used to produce strong intrathalamic contrast, which allows delineation of the intermedullary lamina and isolation and segmentation of the central thalamic (CL) volume. In some examples, the image resolution (i.e., voxel size) is 1 mm or better and/or isotropic (e.g. equal size for all three voxel dimensions), and/or the imaging volume covers the whole brain of the human subject.

In some examples of this technology, the resulting WMn images are processed (e.g., by the surgical computing device 14) to segment (or define spatial boundaries of) structures within the thalamus of the human subject. One exemplary approach for this segmentation is to use the Thalamus Optimized Multi-Atlas Segmentation (THOMAS) algorithm as disclosed in Su et al., “Thalamus Optimized Multi Atlas Segmentation (THOMAS): fast, fully automated segmentation of thalamic nuclei from structural MRI,” Neuroimage. 2019 Jul. 1; 194:272-282, which is incorporated herein by reference in its entirety, although other methods for segmentation can also be used. The THOMAS algorithm segments multiple thalamic nuclei on each of the brain image volumes.

Another exemplary approach to segmentation in accordance with the disclosed technology is to use a single-atlas method for warping masks that label the CL and VPM nuclei from a template brain volume to the individual image volume of interest. Since the THOMAS algorithm does not identify or segment the CL and VPM nuclei, this second stage of thalamic segmentation may be performed in some examples of this technology. There are several nuclei identified by the THOMAS algorithm that may represent “target avoidance regions” such as the CM nucleus, but the primary target activation region is the CL nucleus, which is identified on both sides of the brain for each individual image volume of interest using the single-atlas method of this second step of thalamic segmentation.

In step 502, the surgical computing device 14 models one or more fiber pathways in the segmented brain model generated in step 500. Identification of the location of this confluence of fiber pathways can be optimally achieved, for example, with the use of diffusion tensor imaging (DTI) according to Edlow et al., “Neuroanatomic Connectivity of the Human Ascending Arousal System Critical to Consciousness and Its Disorders,” J. Neuropathol. Exp. Neurol. 71 (6): 531-46 (2012), which is incorporated herein by reference in its entirety. In some examples, the surgical computing device 14 acquires diffusion weighted images in a manner consistent with DTI processing to cover the whole brain of the human subject with isotropic resolution at 2 mm voxel dimension or better. The diffusion weighted images can be acquired using imaging sequence parameters that produce high image quality and signal-to-noise ratio.

These diffusion weighted images can then be processed using DTI fiber tractography, in which a specific fiber tract is defined according to a seed region where fiber pathways originate, a “filter region” through which tracked fibers must pass, and optionally an endpoint region where tracked fibers may reach. Using fiber tractography methods, the surgical computing device 14 defines the CL/DTTm fiber bundle, which originates at the pedunculopontine nucleus (PPN), passes through the CL nucleus of the thalamus, and ends in the frontal or parietal brain.

In step 504, the surgical computing device 14 generates a position and orientation in the segmented brain model for at least one electrode that has a plurality of contacts. The combination of the spatial location of the lateral wing of the CL as the target point, and the direction of the CL/DTTm, defined by the DTI and fiber tractography, yields a dominant axis of the CL/DTTm fiber bundle in three dimensions, as well as a target electrode position and orientation. More specifically, the orientation of the electrode is based on the orientation of the contacts of the electrode, which corresponds with the determined dominant axis of the CL/DTTm fiber bundle. The surgical computing device 14 also determines a surgical trajectory of electrode insertion to achieve the target position and orientation. Accordingly, the target electrode position and orientation guide DBS lead localization, as described and illustrated in more detail below.

Optionally, the electrode position can further be generated based on data stored on surgical computing device 14 for identifying areas for implantation to provide selective activation of the subject's thalamus. In one embodiment, the segmented brain model is registered to a brain model atlas to identify anatomical nuclei in the segmented brain model in order to identify the electrode position. The registration may be performed using a technique such as symmetric normalization, for example, although other techniques can also be used.

In step 506, the surgical computing device 14 produces a stimulation map. The stimulation map is produced using the segmented model of the subject's central thalamus. The electrode position is used to apply a modeled stimulus in order to generate the stimulation map to identify the fiber pathways that are activated as a result of applying the model stimulus. In some examples, the activation fiber bundles, and/or avoidance fiber bundles, are rendered using biophysical modeling as applied to 3D fiber trajectories developed from DTI. Modeling this interaction is performed by first calculating the electric field produced in the brain as a function of the electrode location and stimulation settings, and second by predicting activation based on the voltage values along each tract or within each nucleus.

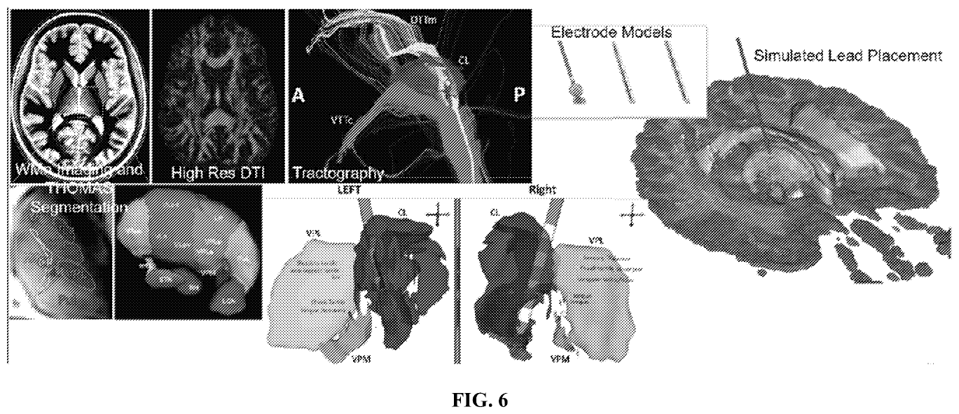

Referring to FIG. 6, methods used for image-guided surgical planning to facilitate vector-based targeting of a human central thalamus to guide deep brain stimulation are illustrated. In particular, FIG. 6 illustrates an overview of methods used for image-guided surgical planning of CT-DBS including segmentation of thalamus and thalamic nuclei utilizing MRI imaging with enhanced thalamic contrast and automated segmentation. In this example, WMn imaging is used with the THOMAS plus CL-VPM automated segmentation thalamic segmentation algorithms to define target and avoidance nuclei, DTI with tractography is used to define target and avoidance fiber tracts, and electrode and biophysical modeling of neuronal activation is used to identify electrode position and orientation and surgical trajectory.

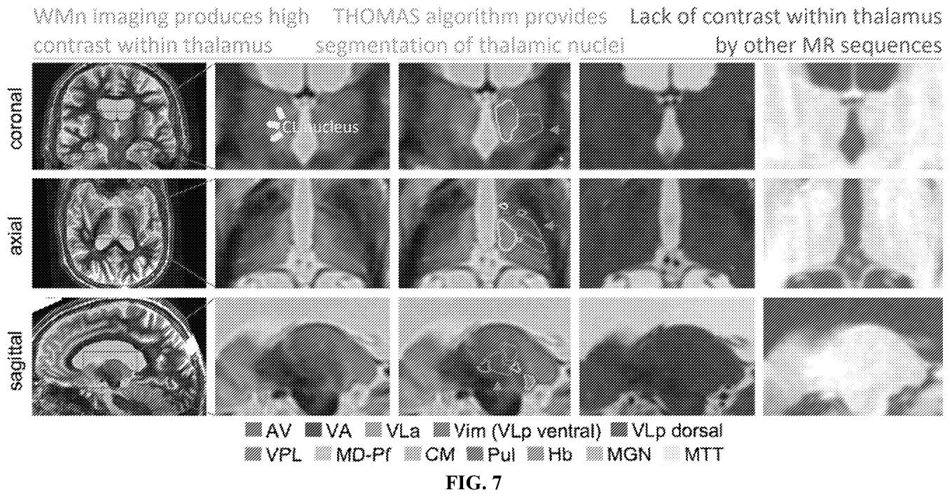

Referring to FIG. 7, WMn imaging showing contrast within a thalamus to allow identification of individual thalamic nuclei is illustrated. In this example, WMn imaging shows contrast within the thalamus to allow clear identification of individual thalamic nuclei including visual evidence of CL nucleus as well as sufficient contrast to permit automated segmentation of 14 thalamic nuclei using the THOMAS algorithm. Accordingly, WMn imaging with high contrast within the thalamus of the human subject facilitates improved segmentation of thalamic nuclei using the THOMAS algorithm, which may not be possible using other magnetic resonance sequences that provide no or reduced contrast within the thalamus.

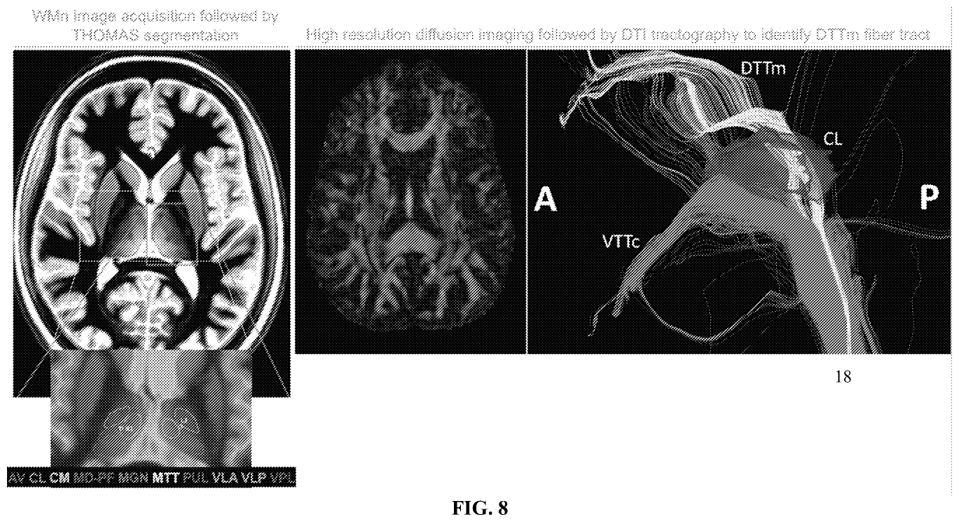

Referring to FIG. 8, a combination of WMn and DTI imaging that provides both target and avoidance nuclei, as well as target and avoidance fiber tracts, is illustrated. The target and avoidance nuclei and fiber tracts are used to define vector-based targeting that takes into account both the position and the trajectory (i.e., orientation) of the DBS leads (e.g., electrode contacts) relative to the target projections from the nucleus and the fiber bundles emanating from this nucleus. Accordingly, the vector-based targeting of this technology combines the three-dimensional model of the thalamic nucleus and the model fibers projecting from the nucleus to target structures in the frontal cortex and striatum, for example, of a human subject. High resolution diffusion imaging followed by DTI tractography is used in this example to identify the DTTm fiber tract, which facilitates determination of the dominant axis of the DTTm fiber tract and corresponding orientation of electrode contacts and surgical trajectory.

Referring back to FIG. 5, in step 508, the surgical computing device 14 optionally determines whether the electrode position, contact orientation, and surgical trajectory are satisfactory. The determination regarding the electrode position and contact orientation can be based in part on the stimulation map produced in step 506 and whether the electrode position is ideal for selectively activating the central thalamus fibers of the subject so that central lateral nucleus and medial dorsal tegmental tract fiber pathway activation in the subject is maximized and centromedian-parafascicularis fiber pathway activation in the subject is minimized. The determination regarding the surgical trajectory may be based on particular anatomical structures of the human subject, such as one or more lesions that may be desirable to avoid during insertion of the electrode(s), for example. The one or more lesions are in one or more of the central thalamus, cerebral cortex, or striatum. In some examples, the determination in step 508 can be automated, such as when the surgical trajectory impacts a brain lesion, and in other examples, the determination in step 508 can be based on manual observation and surgeon input to the surgical computing device 14. If the surgical computing device 14 determines that one or more of the position, orientation, or surgical trajectory is unsatisfactory, then the No branch is taken back to step 504.

In a subsequent iteration of steps 504-506, the surgical computing device generates another electrode position and/or orientation and/or another surgical trajectory that remains in substantial alignment with the dominant axis of the CL/DTTm fiber bundle, but improves activation or avoidance, and/or avoids lesion(s), for example. In some examples, navigation around lesions within the thalamus is achieved by making adjustments with respect to increasing coverage of activation of remaining fibers available in target acquisition structures and avoidance of nearby regions of fibers representing target avoidance structures.

For an illustrative example, modeling fibers surrounding a local thalamic lesion obstructing some of the fibers for target acquisition emanating within the volume of tissue to be stimulated may be problematic. Using the bioelectric field modeling described above with reference to step 506, single or multiple electrodes are virtually placed and activation of each fiber bundle from target acquisition or target avoidance structures is quantitatively assessed based on local positioning and orientation of the electrode(s) and simulated activation under varying combinations of electrode contact geometries (e.g., active cathodes), and stimulation parameters (e.g., amplitude of voltage or current, pulse width of stimulation pulses, frequency of stimulation pulses, phase per contact of stimulation signal). This approach allows the planning of single or multi-electrode systems to navigate placements in brains with large, multifocal lesions.

Referring back to step 508, if the surgical computing device 14 determines that the position, orientation, and surgical trajectory are satisfactory, then the Yes branch is taken to step 510. In step 510, a position and orientation for the one or more electrodes in the subject's central thalamus fibers, surgical trajectory, and electrical stimulus conditions for the electrode(s) are established and used to insert the electrode(s) and selectively activate the central thalamus fibers of the subject so that central lateral nucleus and medial dorsal tegmental tract fiber pathway activation in the subject is maximized and central median parafascicularis fiber pathway activation in the subject is minimized based on the simulation map produced in step 506.

Accordingly, the electrode(s) are positioned in the subject's central thalamus fibers such that the contacts of the electrode(s) are in substantial alignment with the orientation of the dominant axis of the CL/DTTm fiber bundle and so as to avoid lesions in some examples. Optionally, stimulation induced voltages are shaped to achieve selective activation of the target fiber pathways or nuclei while avoiding non-target pathways or nuclei. Shaping is achieved through the implantation of one or more DBS leads in each hemisphere, as well as selection of stimulation settings, including those where both inter- and intra-lead stimulation can be applied.

The exemplary method may be employed in pre-operative, intra-operative, and post-operative settings. Pre-operative planning may be employed to determine locations, orientations, and trajectories to implant the electrodes/leads in each brain hemisphere to have the highest likelihood of activating the target structures while avoiding other structures. During pre-operative planning, a wide range of range of DBS lead positions, orientations, and trajectories are explored. The parameter space includes a 6 degree of freedom problem in terms of spatial transformations, and 7 degrees of freedom for directional DBS leads. The described methods allow for determining locations and orientations to implant the electrodes, such as electrodes 32 to selectively activate the central thalamus fibers of the subject so that central lateral nucleus and medial dorsal tegmental tract fiber pathway activation in the subject is maximized and central medial parafascicularis fiber pathway activation in the subject is minimized.

The exemplary method may also be employed intra-operatively to further determine if the applied activation is on target during execution of the pre-operative plan. Information gathered intra-operatively, such as feedback from sensors 40, is used to assess the degree to which the pre-operative plan is being followed. This data is recorded and stored in the subject model on surgical computing device 14. One or more sensors 40 are temporarily implanted in the subject to record neural activity that could indicate whether the pre-operative plan is being executed. Intra-operative imaging (MRI, CT, endoscopy) using imaging device 16 may also be employed to confirm the lead position.

Additionally, the exemplary method may be utilized in post-operative planning. Post-operative planning may be utilized to program the stimulator, such as stimulus signal generator 38, to provide stimulation to the subject to provide a therapeutic benefit. Post-operative imaging (MRI or CT) using imaging device 16 is used to confirm the actual DBS lead locations and orientations, such as electrodes 32, in each hemisphere. This imaging is co-registered with the pre-operative imaging in the subject model stored on surgical computing device 14. At this point, the lead locations are fixed and cannot be changed without an additional surgery. Therefore, the electrical stimulation conditions, as described above, such as which electrodes to activate as anodes or cathodes and what waveforms to use to achieve target activation with minimal spill-over into other structures may be adjusted. Simulations are used to systematically explore this parameter space and recommend stimulation settings for stimulus signal generator 38, such as a pulse generator.

The system 12 will further allow the post-implantation location of the electrode(s) to be determined instantly to allow for accurate post-implantation titration of behavioral effects and annotation of positive and negative behavioral effects to customize the system for programming of electrical current for an individual subject. The system 12 will also allow for post-implantation titration of electrical evoked activity when used in conjunction with high density EEG.

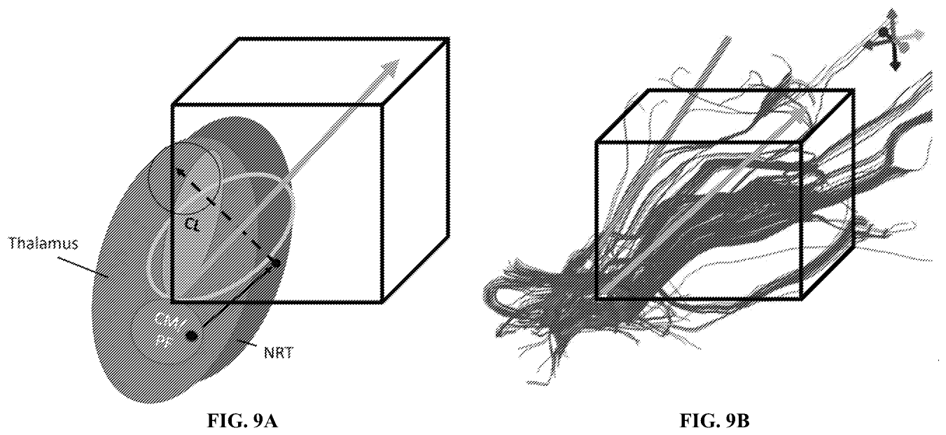

Referring to FIGS. 9A and 9B, a conceptual overview showing placement of a vector in a three-dimensional collection of fibers to be adjusted for bulk activation of fibers of the CL/DTTm structure is illustrated. The vector is placed via initial lead placement in virtual space using MR imaging to select a skull entry location and tip location in substantial alignment with a determined dominant axis of the CL/DTTm fiber bundle, estimation of activation of target and avoidance structures, and iterative adjustment of lead trajectory and tip location until at least one electrode can achieve objectives. Accordingly, the vector in FIGS. 9A and 9B represents an orientation of electrode contacts in three-dimensional space that substantially corresponds with a dominant axis of the CL/DTTm fiber bundle and is located and oriented to yield satisfactory target activation and target avoidance.

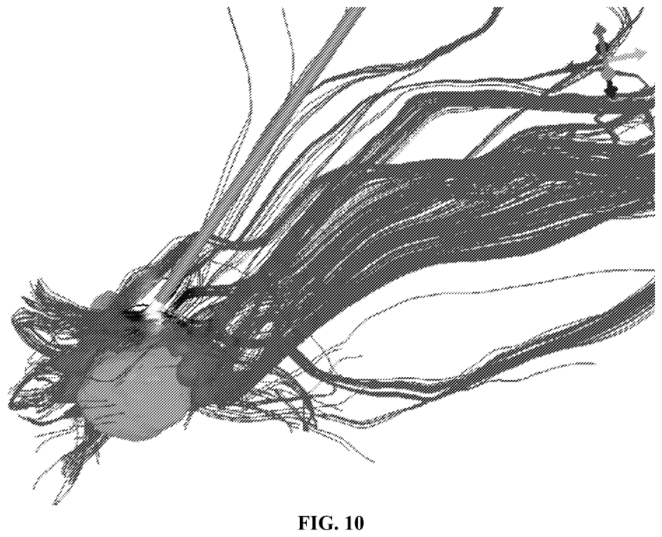

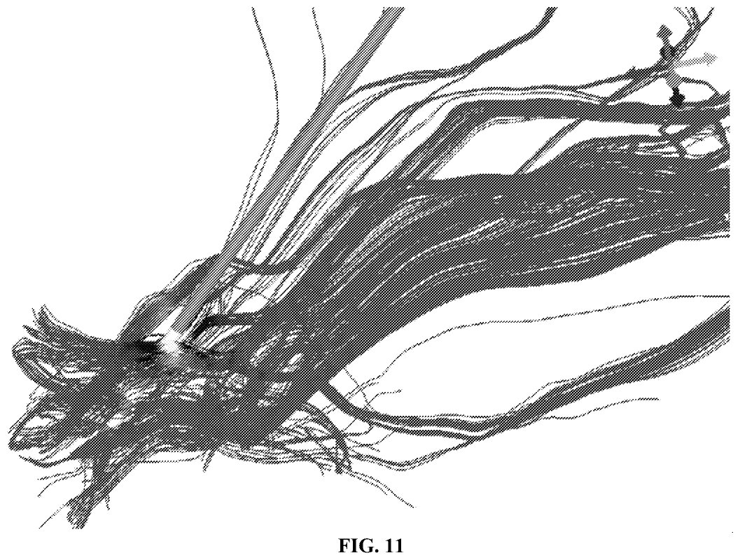

Referring to FIG. 10, a volumetric rendering of two thalamic nuclei (activation target) and centromedian (avoidance target), target DTTm fiber bundle, and a DBS lead with active electrodes is illustrated. In this example, the two thalamic nuclei (CL-blue (activation target)) and centromedian (pink (avoidance target)) of a target DTTm fiber bundle (purple) and DBS leads with active electrodes (gray and white) are illustrated along with an applied electric field (yellow) that activates particular fibers. Referring to FIG. 11, another volumetric rendering of the two thalamic nuclei of FIG. 10 with isolation of fibers activated by applied electric field is illustrated. In this example, the isolated activated fibers are indicated in yellow.

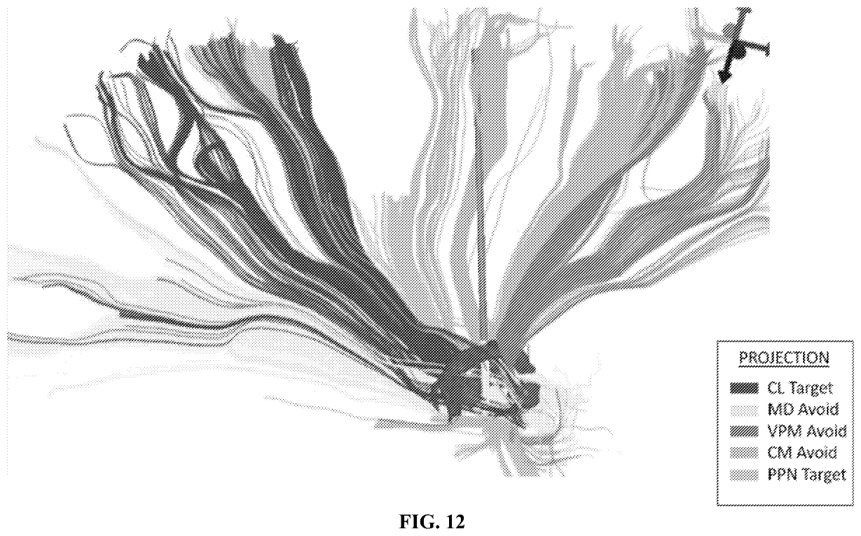

Referring to FIG. 12, multiple target activation and avoidance pathways within the human central thalamus are illustrated. In this particular example, the CL and PPN are target fiber pathways and the MD, VPM, CM are avoidance fiber pathways, although other pathways can be target and/or avoidance fiber pathways in other examples.

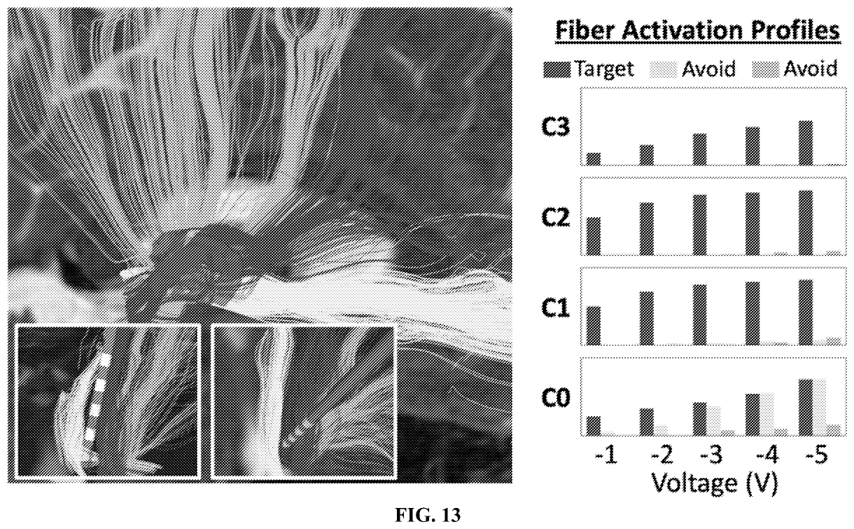

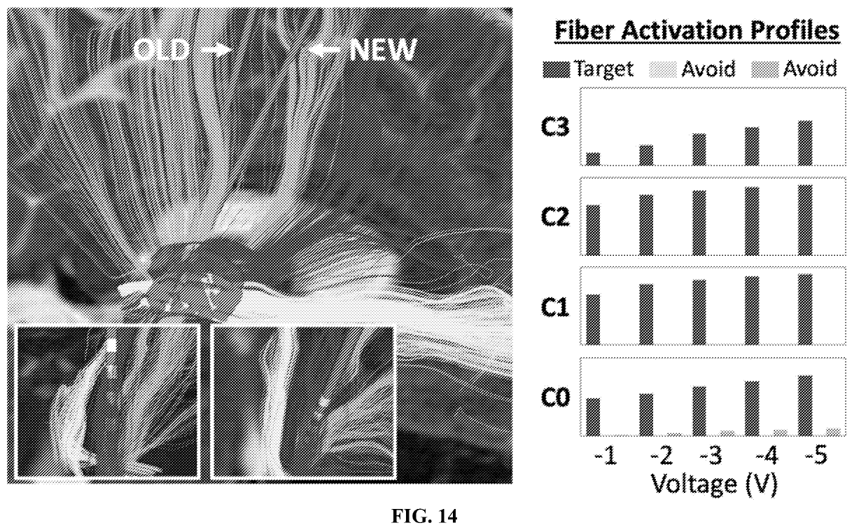

Referring to FIG. 13, fiber activation profiles including histograms of percentage activation of target activation and target avoidance regions for a generic thalamic model system are illustrated. The illustrated histograms in this example show the percentage activation of activation targets (blue) and the percentage activation of avoidance targets (yellow, green) for the generic thalamic model system. Referring to FIG. 14, changes in fiber activation achieved with adjustment of electrode position from that illustrated in FIG. 13 are illustrated. The electrode position is adjusted between FIG. 13 and FIG. 14 in accordance with the disclosed technology such that the orientation of the contacts of the electrodes are substantially aligned with the dominant axis of the fiber bundle, resulting in improved target activation and reduced activation of avoidance targets/regions.

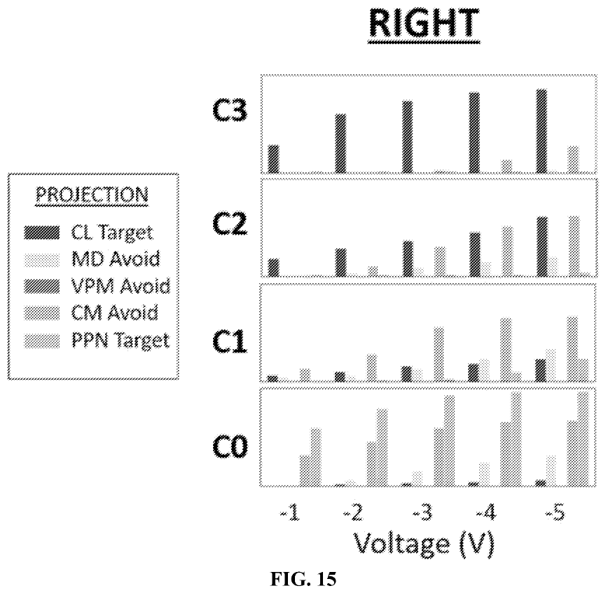

Referring to FIG. 15, human thalamic imaging data from a human subject with TBI including the percentage activation of CL and PPN targets and other thalamic nuclei for avoidance (VPM, CM, MD) is illustrated. As shown in FIG. 15, the activation of the activation targets is increased, and the activation of the avoidance targets is reduced, via the four contacts of an exemplary electrode according to the technology described and illustrated herein.

### Examples

The present description is further illustrated by the following examples, which should not be construed as limiting in any way. In one example, the lateral portion (‘wing’) of the central lateral thalamic nucleus and its associated fiber bundle, the dorsal tegmental tract, medial component (DTTm), CL/DTTm-DBS were selected as the target for activation in six human subjects (ages 23-60, 3-18 years post-injury), with five of the six subjects completing testing, as illustrated below in Table 1 along with corresponding demographically-adjusted scores.

TABLE 1

| | Patient | Patient | Patient | Patient | Patient |
|---|---|---|---|---|---|
| | 1 | 2 | 3 | 4 | 5 |
| Age | 39 | 60 | 22 | 30 | 28 |
| Yrs since Injury | 18 | 3 | 4 | 10 | 9 |
| Trails B Pre-Surg | 153 | 171.7 | 39 | 42.6 | 165.61 |
| Trails B End Treatment | 129.7 | 82.9 | 29 | 32.4 | 95.19 |
| % Change Trails B | −15.2288 | −51.7181 | −25.641 | −23.9437 | −42.26637 |
| | Mean: | −31.7596 | | | |
| Trails A Pre-Surg | 62 | 85.9 | 22.1 | 18.7 | 61.6 |
| Trails A End Treatment | 41.5 | 45.9 | 16.5 | 14.7 | 44.72 |
| % Change Trails A | −33.0645 | −46.5658 | −25.3394 | −21.3904 | −27.4026 |
| | Mean: | −30.7525 | | | |
| QoL Attention Pre-Surg | 19 | 6 | 14 | 10 | 14 |
| QoL Attention End | 30 | 10 | 21 | 23 | 27 |
| Treatment | | | | | |
| % Change Qol Attention | 57.89474 | 66.65667 | 50 | 130 | 92.857143 |
| | Mean: | 79.48371 | | | |
| QoL Executive Function | 36 | 20 | 25 | 26 | 24 |
| Pre-Surg | | | | | |
| QoL Executive Function | 43 | 20 | 32 | 43 | 39 |
| End Treatment | | | | | |
| % Change of QoL Exec | 19.44444 | 0 | 28 | 53.57143 | 62.5 |
| Function | | | | | |
| | Mean: | 32.70317 | | | |
| Demographically | Patient | Patient | Patient | Patient | Patient |
| Adjusted T-Scores | 1 | 2 | 3 | 4 | 5 |
| | TMT-B | | | | |
| Trails B Pre-Surg | 22 | 33 | 62 | 62 | 22 |
| Trails B End Treatment | 22 | 50 | 71 | 71 | 35 |
| \|Change over time: | 0 | 17 | 9 | 9 | 13 |
| | | | | Average: | 9.6 |

TMT-A

| | | | | | |
|---|---|---|---|---|---|
| Trails A Pre-Surg | 21 | 20 | 49 | 57 | 22 |
| Trails A End Treatment | 30 | 38 | 55 | 71 | 31 |
| \|Change over time: | 9 | 18 | 17 | 14 | 9 |
| | | | | Average: | 13.4 |

The CL/DTTm targeting was implemented based on positioning of stimulation electrodes in the intended location, and adjusting the orientation of the electrodes to optimize stimulation of the intended CL/DTTm fiber bundle, according to the imaging, thalamic segmentation, and predictive biophysical models estimating activation of projection fibers described above in order to meet the need for precise and accurate location of the vector representing the CL/DTTm target in the human subjects. As the primary efficacy endpoint, Part B of the Trail Making Test (TMT-B) was selected, based on the well-established relationship between diffuse axonal injury (DAI) produced by msTBI and persistent disabilities in executive attention and controlled information processing speed.

All subjects had safe bilateral implantation of electrodes, with position and orientation guided by subject-specific imaging to target CL/DTTm. Five subjects completed the study, which included a two-week stimulation titration phase and a three-month open label treatment phase. All five subjects exceeded the pre-selected primary outcome benchmark of 10% improvement in completion time on TMT-B, from pre-surgical baseline to the end of the treatment phase (showing 15%, 24%, 26%, 42%, and 52% improvement), as explained in more detail below.

For each subject white-matter-nulled magnetization-prepared rapid acquisition gradient echo (WMn-MPRAGE) and DTI MRI data was obtained for use in a dedicated processing pipeline. In addition to a conventional scan protocol used for clinical pre-surgical DBS planning, subjects were scanned with a WMn-MPRAGE protocol and a DTI protocol, on a 3T GE MR 750 scanner using a 32-channel head coil. WMnMPRAGE image volumes were acquired using the following parameters: 3D MPRAGE sequence, coronal orientation, TE 4.7 ms, TR 11.1 ms, TI 500 ms, TS 5000 ms, views per segment 240, FA: 8°, RBW+/−11.9 kHz, spatial resolution 1 mm isotropic, 220 slices per volume k-space ordering; 2D radial fanbeam, ARC parallel imaging acceleration: 1.5×1.5. DTI image volumes were acquired using the following parameters: 2D diffusion-weighted single-shot spin-echo echo planar imaging (EPI) sequence, axial orientation, TE 74 ms, TR 8000 ms, RBW+/−250 kHz, diffusion directions: 60, diffusion weighting (b-value): 2500 s/mm\hat{ }2, spatial resolution 2 mm isotropic, 70 slices per volume, parallel imaging acceleration: 2, scan time 11 min. WMnMPRAGE and DTI image volumes were visually inspected to ensure that scans were of sufficient quality for analysis and were not corrupted by motion artifact.

Each subject's WMn images were then processed using the THOMAS automated thalamic segmentation algorithm and, because the THOMAS algorithm did not include the central lateral nucleus as a default subnuclear structure, the CL boundary was identified using a single-atlas segmentation method that employed a CL atlas derived by manual segmentation by an expert neuroradiologist from the THOMAS template, an extremely high quality WMn image formed by non-linear registration and averaging of 20 WMn volumes.

More specifically, whole-brain WMnMPRAGE volumes were processed with the THOMAS thalamic segmentation tool with no preprocessing. The volumes of 12 lateralized structures were segmented and extracted in each hemisphere of the brain: whole thalamus, ten thalamic nuclei (anteroventral [AV], centromedian [CM], lateral geniculate nucleus [LGN], mediodorsal [MD], medial geniculate nucleus [MGN], pulvinar [Pul], ventral anterior [VA], ventral lateral anterior [VLA], ventral lateral posterior [VLP], and ventral posterolateral [VPL]), and one adjacent epithalamic structure, the habenula (Hb). THOMAS segments the whole thalamus separately from the thalamic nuclei; this whole thalamus encompasses all these preceding structures, as well as the mammillothalamic tract and some additional unlabeled thalamic areas (i.e., between segmented thalamic nuclei).

In addition to THOMAS segmentation, the CL and VPM nuclei in each hemisphere were segmented using a single-atlas segmentation approach. This utilized manually-segmented CL and VPM nuclei, performed by a single expert neuroradiologist (TT) on the THOMAS template, which is an extremely high quality WMn brain volume formed by carefully registering and averaging 20 WMn volumes. The CL and VPM single-atlases obtained this way were non-linearly warped to the WMn volumes of individual subjects, and CL and VPM boundaries were finalized by trimming away any CL and VPM voxels which overlapped with THOMAS nuclei.

In other words, THOMAS segmentations were allocated higher priority than CL and VPM segmentations—the rationale for this being that the THOMAS segmentations (obtained with a multi-atlas approach) are more accurate than the CL and VPM single-atlas segmentations. Thus, The CL and VPM segmentations were prevented from overlapping with THOMAS nuclei by giving priority to the latter. However, in alternative implementations, it may be preferred to prioritize the CL and/or VPM segmentations over the THOMAS segmentations. The DTI images were analyzed to obtain tractography models for fibers emanating from CL and other neighboring thalamic nuclei generated by the THOMAS algorithm.

The CL nucleus and the fiber bundle of axons emanating from this region the dorsal tegmental track medial (DTTm) were then targeted based on several operational distinctions delineating the boundaries of the intended target region. Based on known monosynaptic connections determined in prior physiological and anatomical studies, stimulation cell bodies and axonal regions with reciprocal connections of the ‘lateral wing’ of CL and prefrontal/frontal cortical regions include anterior cingulate (area 24), premotor, pre-supplementary motor/supplementary motor area (area 6), and dorsomedial prefrontal cortex including frontal eye fields (areas 8 and 9) was sought. In addition, placements of electrodes were planned to stimulate fibers emanating from the paralaminar region of medial dorsalis nucleus (plMD) which have strong projections to dorsal lateral prefrontal cortex (area 46). Collectively, the primary monosynaptic projections in the expected stimulation regions span the medial prefrontal/frontal regions with some extension over the lateral convexity of the frontal cortex.

To guide electrode placements to achieve this targeting of CL/DTTm, finite element models and biophysical modeling of fiber activation using model electrodes targeted in subject brain space adjusted by safety and angle of entry point were then used in consideration of local blood vessel anatomy. Lead and electrode placement and orientation were adjusted to simultaneously maximize activation of the CL/DTTm fiber tract and minimize activation of off-target fibers.

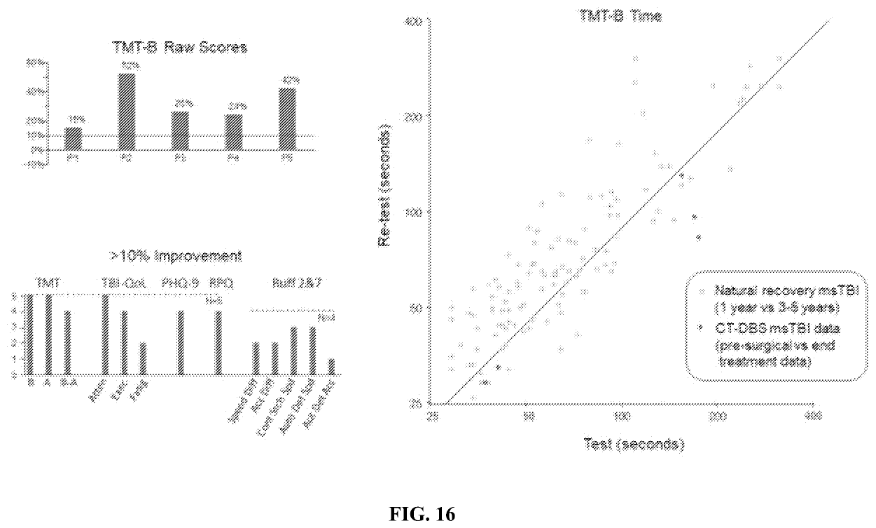

Five subjects completed the full study design that included a two-week stimulation titration phase (TP) and a three-month open label (OL) treatment phase. As illustrated in FIG. 16, all five of these subjects met the pre-selected primary outcome benchmark of a greater than 10% improvement in in completion time on TMT-B from pre-surgical baseline to the end of the TP (average improvement 31.75; min 15%, max 52%). The range of improvements spanned 15% to 52%. The greatest percentage improvements were seen in the subjects with greatest initial deficits. However, even subjects whose baseline performance was in the upper range of normal demonstrated a greater than 20% improvement in performance times.

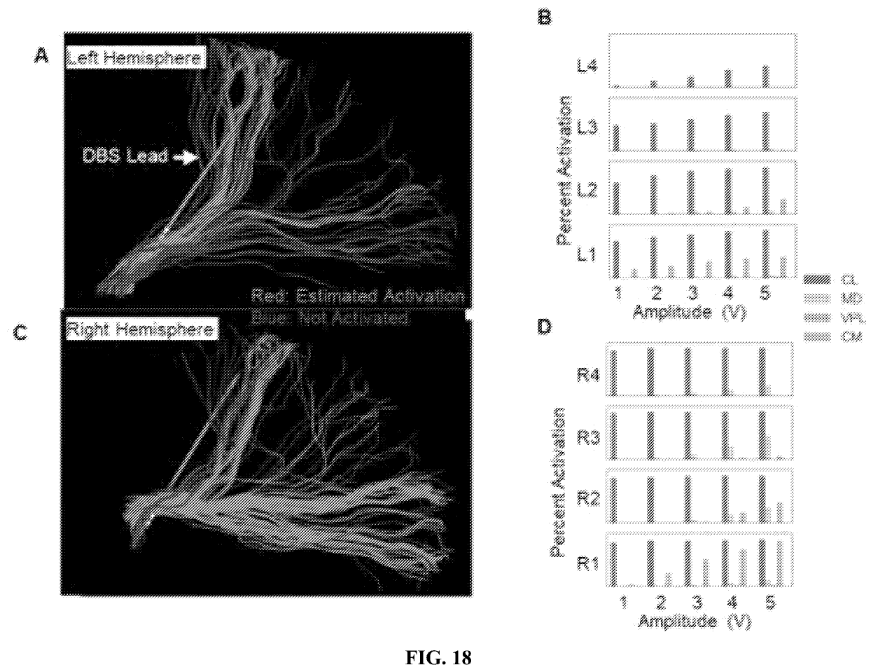

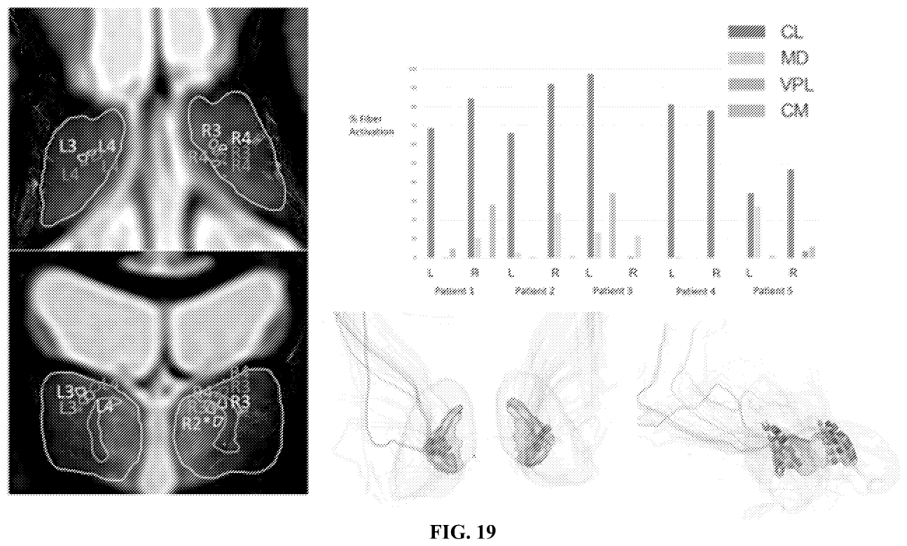

In more granular detail, FIGS. 18 and 19 illustrate exemplary approaches to target acquisition from representative human subjects along with activation results from both hemispheres. Images in the middle top row of FIG. 18 identify the location of active electrode contacts in patient 3 displayed on coronal WMn images with CL volume shown in yellow (blue outlines for two left hemisphere, L3, L4, and two right hemisphere, R3, R4 contacts). Light red markings in the coronal images delineate the passing DTTm fibers and show their spatial proximity to the active contacts. At the left and right of the top row are illustrations of the CL/DTTm fiber bundle activation achieved within the left and right hemisphere. For this subject, combined activation of the four active contacts achieved an 81% activation of CL/DTTm fibers within the left hemisphere and a 78% activation of these fibers within the right hemisphere. The histograms plotted in the lower middle row show the percentage of activation for CL/DTTm, MD, VPL, and Cm fibers. For most contacts CL/DTTm fiber activation dominated the range of current amplitudes modeled for single contact monopolar activation. These histograms formed the basis for titration testing used to establish electrode contact geometry and stimulation parameters in the treatment trial.

Across the patient subjects, a similar profile for modeled activation of CL/DTTm was obtained with most electrode placements resulting in a dominant activation of these fibers. However, in patient 3, electrode contacts within the right hemisphere failed to activate modeled CL/DTTm fibers (0.5% predicted activation). For most subjects, active contacts produced modeled activation of modeled CL/DTTm fibers with limited involvement of the avoidance fibers.

To compare electrode placements across the five human subjects, a synthetic atlas was developed to organize all patient electrode placements within a single common space. FIG. 19 illustrates the placements of active contacts for each subject in the common synthetic atlas space is illustrated. FIG. 19 demonstrates a tight clustering of active contacts for left hemisphere electrodes around the emergence of the CL/DTTm fibers exiting the CL nucleus boundary (red light marks), but placements of the active right hemisphere electrode contacts showed greater variability. This difference is likely influenced by shifts in brain volume induced by loss of cerebral spinal fluid during the procedures as the right hemisphere electrodes were typically placed after the left (⅘ subjects). Also illustrated in FIG. 19 are top and angled lateral views of the left and right electrodes illustrating the tight clustering of placements within the left hemisphere and the relationship of the CL/DTTm fiber bundle along with the relative activation percentage for CL/DTTm and the avoidance fibers from MD, VPL, and Cm.

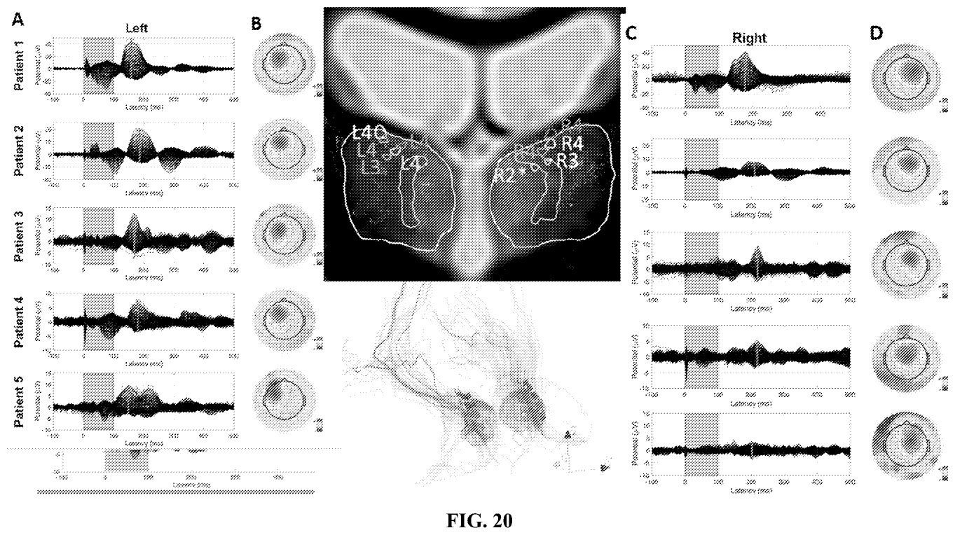

Referring to FIG. 20, cortical evoked potentials obtained across a 128 channel EEG array for activation across two active contacts using a 2 Hz duty cycle of stimulation is illustrated. Each row of FIG. 20 shows cortical evoked potential time tracings from all 128 channels superimposed. For both hemispheres, these evoked responses typically demonstrated an initial positive deflection peaking at ˜200 ms after the stimulation pulse followed in most subjects by second and sometimes third shallower peak activations, settling of the evoked response to flat baseline typically occurred within ˜1 second. Topographical plots indicating the spatial variation in depth of evoked response at the time of the peak (˜200 ms, see red lines) indicates that the strongest response appears within the frontal regions of the ipsilateral hemisphere between the medial and lateral regions.

As illustrated in FIG. 20, a more reproducible localization, depth of modulation and timing of peaked amplitude response is present across subjects in the left hemisphere. Comparing these finding to those obtained from the synthetic atlas in FIG. 19, a correspondence of the tighter cluster of electrode contact positions in the left electrode leads suggest the inter-subject consistency of activating the same fiber system is greater in the left hemisphere. Right-sided electrode placements showed greater variance in tip placement than at the top contacts used for activation. In some examples of this technology, intraoperative measurements of the evoked potentials can be used to facilitate or adjust electrode position, orientation, or one or more other parameters of electrode activation based on assessment of localization, depth of modulation, and/or timing of cortical evoked responses. Such implementations can employ methods of measurement of the electric activity of the brain (e.g. surface electroencephalography electrodes, subdural grid or strip electrodes, or indwelling tissue electrodes), memory storage in a computer, a method of averaging, and a method of visual display for real-time intra-operative feedback to the neurosurgeon, for example.

Five subjects completed the full study design that included a two-week stimulation titration phase (TP) and a three-month open label (OL) treatment phase. As seen in FIG. 19, all five of these subjects met the pre-selected primary outcome benchmark of a greater than 10% improvement in in completion time on TMT-B from pre-surgical baseline to the end of the TP (average improvement 31.75; min 15%, max 52%). The range of improvements spanned 15% to 52%. The greatest percentage improvements were seen in the patients with greatest initial deficits (i.e., patients 2 and 5 shown above in Table 1). However, even subjects whose baseline performance was in the upper range of normal (e.g., patients 3 and 4) demonstrated a greater than 20% improvement in performance times.

To further assess these results, two additional comparisons were conducted. The Trail making test is among a set of neuropsychological tests that have been demographically adjusted for a range of variables as part of the Halstead-Reitan Neuropsychological Test Battery. Using the demographically adjusted T-scores applied for each subject's specific characteristics it was found that the average performance improvement across all subjects on TMT-B is 9.6 (as shown in Table 1), which is 0.98 standard deviations (T scores are normalized so that one standard deviation equals 10 points).

Second, to estimate the likelihood of such changes in TMT-B times occurring spontaneously, the measurements to a database of longitudinal measurements of TMT performance obtained from 118 msTBI subjects followed at 1 and 3-to-5 year timepoint (subjects drawn from a subset of those included in a published study of Dikmen et al., “Outcome 3 to 5 Years After Moderate to Severe Traumatic Brain Injury,” Arch Phys Med Rehabil Vol 84, October 2003 (“Dikmen”), which is incorporated herein by reference in its entirety). For the primary outcome measure, TMT-B, improvements reflect changes in the central executive components of working memory and set-switching collected under the term ‘cognitive-flexibility”; improved TMT-B performance likely indexes functional changes in prefrontal, parietal cortical neurons (REFS) linked to CL/DTTm electrical stimulation.

FIG. 16 shows a scatterplot of 1 year versus 3-5 year TMT-B performance in the individual Dikmen subjects (blue filled circles) and the five subjects of the instant example (orange filled circles). As seen in the figure the five subjects are distributed along the lower edge of the cloud of the distribution of longitudinal changes in the Dikmen subjects. The observed set of 5 longitudinal changes in TMT-B times found in our study (15 to 52% faster) differs substantially from the longitudinal changes in the Dikmen dataset (mean change, 4% slower): Kolmogorov-Smirnov test, p<0.005 [0.0041. Note also that the three-month time course of our study (compared with the 3-to-5 year interval in Dikmen) and the starting point of three or later years after injury make this a conservative comparison. In addition, as seen in FIG. 16, with reference to the line of identical performance of the test (y=x) on each measurement, the Dikmen subjects tended toward worsening performance over time (with more data points above the line).

The subjects of this example also showed improved performance on TMT-A, which primarily tests search speed and may be also linked to frontostriatal function. In addition, the derived measure B-A, which probes executive control showed improvement. Compared to the test-retest data of Dikmen, the changes observed were significant (TMT-A: 21 to 47% faster in present study, mean change 6% slower in Dikmen, Kolmogorov-Smirnov test, p<0.001 [0.00057 The demographically-adjusted average performance improvement across all subjects on TMT-A is 13.4 (as shown above in Table 1), indicating a greater than one standard deviation improvement. Collectively the comparison results in this example demonstrate that the faster completion times in TMT-B, TMT-A, and B-A in the CL/DTTm subjects of this example are highly unlikely to be the result of spontaneous test-retest fluctuations.

Additionally, the Ruff 2&7 test was used as an additional performative measure to further evaluate attentional function. One subject's baseline assessment was lost due to test administration error. This measure also showed broad improvements across the four subjects with improved speed difference and accuracy difference seen in all four, improvements in controlled search speed and auto detection speed in three of four subjects completing the full set of testing.

The pre-selected secondary measure TBIQOL-Fatigue showed improvement for 2 participants who met the improvement benchmark, 1 remained stable, and two met the benchmark for decline. Four of the five subjects also showed a greater than 10% improvement on the TBIQol-Executive Function (average improvement 32.7%; min 0, max 62%). The improvements on the TBIQOL-Attention and TBIQol-Executive Function scales reflect self-reported improvements. Despite the short three-month OL phase, two of the four subjects who completed the trial showed a 1-point increase in their Glasgow Outcome Scale Extended (GOS-E) rating from the presurgical baseline to the end of the TP.

Although preferred embodiments have been depicted and described in detail herein, it will be apparent to those skilled in the relevant art that various modifications, additions, substitutions, and the like can be made without departing from the spirit of the application and these are therefore considered to be within the scope of the application as defined in the claims which follow.

What is claimed:

1. A method for vector-based targeting of a human central thalamus to guide deep brain stimulation, the method comprising:
providing one or more electrodes each with a plurality of contacts;
determining a three-dimensional orientation of a dominant axis of a central lateral nucleus dorsal tegmental tract medial component (CL/DTTm) fiber bundle of the human subject;
positioning the plurality of contacts of the one or more electrodes in the human subject's central thalamus fibers in substantial alignment with the determined three-dimensional orientation of the dominant axis of the CL/DTTm fiber bundle; and
applying an electrical stimulus to the positioned plurality of contacts of the one or more electrodes to selectively activate the central thalamus fibers of the human subject, wherein the positioning and the applying are carried out to maximize activation of a central lateral nucleus and medial dorsal tegmental tract fiber pathway in the human subject and to minimize activation of a centromedian-parafascicularis fiber pathway in the human subject.

2. The method of claim 1, wherein a plurality of electrodes are provided.

3. The method of claim 1, wherein 75% to 100% of the central thalamus fibers of the CL/DTTm fiber bundle are stimulated in carrying out the method.

4. The method of claim 1, wherein less than 25% of centromedian-parafascicularis fibers in the centromedian-parafascicularis fiber pathway in the central thalamus are stimulated in carrying out the method.

5. The method of claim 1, wherein 90% to 100% of central thalamus fibers of the CL/DTTm fiber bundle, and less than 10% of centromedian-parafascicularis fibers in the centromedian-parafascicularis fiber pathway in the central thalamus, are stimulated in carrying out the method.

6. The method of claim 1, further comprising:
determining one or more surgical trajectories that substantially avoid one or more lesions, wherein said positioning is further based on the determined surgical trajectories.

7. The method of claim 6, wherein the one or more lesions are in one or more of the central thalamus, cerebral cortex, or striatum.

8. The method of claim 1, wherein the applying an electrical stimulus is carried out at 0.1 to 25.0 milliamps or 0.1 to 10.5 volts, selected independently for each of the one or more electrodes.

9. The method of claim 1, wherein the applying an electrical stimulus is carried out using continuous, intermittent, or periodic stimulation.

10. The method of claim 1, wherein the applying an electrical stimulus is carried out using substantially in-phase or substantially out-of-phase stimulation on each of the one or more electrodes.

11. The method of claim 1, wherein the applying an electric stimulus is ramped up or down at different rates of speed.

12. The method of claim 1, wherein the applying an electrical stimulus is carried out using voltage wave trains having a monophasic or biphasic sine, square, spike, rectangular, triangular, or ramp configuration.

13. The method of claim 1, wherein the applying an electrical stimulus is carried out at one or more frequencies of from 1 Hz to 10 KHz.

14. The method of claim 1, wherein the applying an electrical stimulus is carried out using one or more stimulation programs that are capable of being interleaved in time.

15. The method of claim 1 further comprising:
providing at least one sensor in communication with a brain of the human subject;
determining a state of neuronal activity in the human subject's brain based on data from the at least one sensor; and
adjusting the application of the electrical stimulus based on the determined state of neuronal activity in the human subject's brain.

16. The method of claim 1, further comprising:
imaging the human subject's brain;
segmenting the central thalamus of the human subject's imaged brain to produce a segmented brain model;
ascertaining one or more electrode positions, orientations, or electrical stimulation conditions within the segmented brain model that will maximize activation of a central lateral nucleus and medial dorsal tegmental tract fiber pathway in the human subject and minimize activation of a centromedian-parafascicularis fiber pathway in the human subject; and
producing a stimulation map based on the ascertaining, wherein the stimulation map is used to carry out the positioning and the applying.

17. A method of treating a condition characterized by impaired arousal regulation in a human subject, the method comprising:
selecting a human subject with impaired arousal regulation;
providing one or more electrodes each with a plurality of contacts;
determining a three-dimensional orientation of a dominant axis of a central lateral nucleus dorsal tegmental tract medial component (CL/DTTm) fiber bundle of the selected human subject;
positioning the plurality of contacts of the one or more electrodes in fibers of a central thalamus of the selected human subject in substantial alignment with the determined three-dimensional orientation of the dominant axis of the CL/DTTm fiber bundle; and
applying an electrical stimulus to the positioned plurality of contacts of the one or more electrodes to treat the selected human subject for impaired arousal regulation, wherein the positioning and the applying are carried out to maximize activation of a central lateral nucleus and medial dorsal tegmental tract fiber pathway in the selected human subject and to minimize activation of a centromedian-parafascicularis fiber pathway in the selected human subject.

18. The method of claim 17, wherein a plurality of electrodes are provided with each electrode having a plurality of spaced contacts.

19. The method of claim 17, wherein 75% to 100% of medial dorsal tegmental tract fibers in the medial dorsal tegmental tract fiber pathway in the central thalamus of the selected human subject are stimulated in carrying out the method.

20. The method of claim 19, wherein less than 25% of centromedian-parafascicularis fibers in the centromedian-parafascicularis fiber pathway in the central thalamus of the selected human subject are stimulated in carrying out the method.

21. The method of claim 17, wherein the applying an electrical stimulus is carried out at 0.1 to 25.0 milliamps or 0.1 to 10.5 volts, selected independently for each of the one or more electrodes.

22. The method of claim 17 further comprising:
segmenting the central thalamus in an image of a brain of the selected human subject to produce a segmented brain model;
modelling one or more fiber pathways in the segmented brain model;
generating initial model electrode positions or orientations in the segmented brain model; and
producing a stimulation map based on the modelling and the generating, wherein the stimulation map is used to carry out the positioning and the applying.

23. The method of claim 17, wherein the condition characterized by impaired arousal regulation is selected from the group consisting of brain injury, a neurological degenerative disease, epilepsy, a movement disorder, a post-encephalitis cognitive impairment, a development disorder, a post-hypoxic-ischemic injury cognitive impairment, a neuropsychiatric disorder, post-intensive care unit (ICU) mixed disorder cognitive impairment, and post-ICU adult respiratory distress syndrome.

24. A method for surgical planning involving vector-based targeting of a human central thalamus to guide deep brain stimulation, the method being implemented by one or more surgical computing devices and comprising:
segmenting the central thalamus in an image of a brain of the human subject to produce a segmented brain model;
modelling one or more fiber pathways in the segmented brain model;
determining a three-dimensional orientation of a dominant axis of a central lateral nucleus dorsal tegmental tract medial component (CL/DTTm) fiber bundle of the human subject based on the modelling;
generating initial model positions and orientations in the segmented brain model for one or more electrodes based at least in part on the determined three-dimensional orientation of the dominant axis of the CL/DTTm fiber bundle of the human subject;
producing a stimulation map based on the modelling and the generating; and
identifying a position and orientation for a plurality of contacts of the one or more electrodes in the human subject's central thalamus fibers and electrical stimulus conditions for the positioned and oriented plurality of contacts of the one or more electrodes to selectively activate the central thalamus fibers of the human subject so that activation of a central lateral nucleus and medial dorsal tegmental tract fiber pathway in the human subject is maximized and activation of a centromedian-parafascicularis fiber pathway in the human subject is minimized based on the produced simulation map.

25. The method of claim 24, wherein the generating the initial model positions and orientations within the segmented brain model further comprises:
registering the segmented brain model to a brain model atlas to identify anatomical nuclei in the segmented brain model.

26. The method of claim 25, wherein the registering is performed using symmetric normalization.

27. The method of claim 24, wherein the modelling of the one or more fiber pathways in the segmented brain model is based on diffusion tensor data.

28. A non-transitory computer readable medium having stored thereon instructions for surgical planning involving vector-based targeting of a human central thalamus to guide deep brain stimulation comprising executable code that, when executed by one or more processors, causes the one or more processors to:
segment the central thalamus in an image of the human subject's brain to produce a segmented brain model;
model one or more fiber pathways in the segmented brain model;
determine a three-dimensional orientation of a dominant axis of a central lateral nucleus dorsal tegmental tract medial component (CL/DTTm) fiber bundle of the human subject based on the modelled one or more fiber pathways;
generate an initial model position and orientation in the segmented brain model for each of one or more electrodes based at least in part on the determined three-dimensional orientation of the dominant axis of the CL/DTTm fiber bundle of the human subject;
produce a stimulation map based on the modelled one or more fiber pathways and the generated initial model position and orientation in the segmented brain model for each of one or more electrodes; and
identify a position and orientation for a plurality of contacts of the one or more electrodes in the human subject's central thalamus fibers and electrical stimulus conditions for the positioned and oriented plurality of contacts of the one or more electrodes to selectively activate the central thalamus fibers of the human subject so that activation of a central lateral nucleus and medial dorsal tegmental tract fiber pathway in the human subject is maximized and activation of a centromedian-parafascicularis fiber pathway in the human subject is minimized based on the produced simulation map.

29. The non-transitory computer readable medium of claim 28, wherein the executable code, when executed by the one or more processors, further causes the one or more processors to:
register the segmented brain model to a brain model atlas to identify anatomical nuclei in the segmented brain model to identify the position and orientation for each of the one or more electrodes in the segmented brain model.

30. The non-transitory computer readable medium of claim 28, wherein the registering is performed using symmetric normalization.

31. The non-transitory computer readable medium of claim 28, wherein the modelling of the one or more fiber pathways in the segmented brain model is based on diffusion tensor data.

32. A surgical computing device comprising memory comprising programmed instructions stored thereon and one or more processors coupled to the memory and configured to execute the stored programmed instructions to:
segment a central thalamus in an image of a human subject's brain to produce a segmented brain model;
model one or more fiber pathways in the segmented brain model;
determine a three-dimensional orientation of a dominant axis of a central lateral nucleus dorsal tegmental tract medial component (CL/DTTm) fiber bundle of the human subject based on the modelled one or more fiber pathways;
generate an initial model position and orientation in the segmented brain model for each of one or more electrodes based at least in part on the determined three-dimensional orientation of the dominant axis of the CL/DTTm fiber bundle of the human subject;
produce a stimulation map based on the modelled one or more fiber pathways and the generated initial model position and orientation in the segmented brain model for each of one or more electrodes; and
identify a position and orientation for a plurality of contacts of the one or more electrodes in the human subject's central thalamus fibers and electrical stimulus conditions for the positioned and oriented plurality of contacts of the one or more electrodes to selectively activate the central thalamus fibers of the human subject so that activation of a central lateral nucleus and medial dorsal tegmental tract fiber pathway in the human subject is maximized and activation of a centromedian-parafascicularis fiber pathway in the human subject is minimized based on the produced simulation map.

33. The surgical computing device of claim 32, wherein the one or more processors are further configured to execute the stored programmed instructions to:
register the segmented brain model to a brain model atlas to identify anatomical nuclei in the segmented brain model to identify the position and orientation for each of the one or more electrodes in the segmented brain model.

34. The surgical computing device of claim 32, wherein the registration is performed using symmetric normalization.

35. The surgical computing device of claim 32, wherein the one or more processors are further configured to execute the stored programmed instructions to model the one or more fiber pathways in the segmented brain model based on diffusion tensor data.

36. A system for vector-based targeting of a human central thalamus to guide deep brain stimulation, the system comprising:
the surgical computing device of any one of claims 32-35;
an imaging device operationally coupled to the surgical computing device;
one or more electrodes each comprising a plurality of contacts; and
an electrical stimulator coupled to the surgical computing device and the one or more electrodes to permit electrical activation of the one or more electrodes based on instructions from the surgical computing device.

37. The system of claim 36, wherein the one or more electrodes comprise a plurality of electrodes.

38. The system of claim 36, wherein the electrical stimulator is capable of electrically activating the one or more electrodes to apply an electrical stimulus at 0.1 to 25.0 milliamps or 0.1 to 10.5 volts, selected independently for each of the one or more electrodes.
